(19) United States
(12) Patent Application Publication
(10) Pub. No.: US 2001/0023324 A1
(43) Pub. Date: Sep. 20, 2001

(54) GLUCOSE DETECTOR AND METHOD FOR DIAGNOSING DIABETES

(76) Inventors: ALLAN PRONOVOST, SAN DIEGO, CA (US); JON BOOHER, LAKE FOREST, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/298,398

(22) Filed: Apr. 23, 1999

Related U.S. Application Data
(60) Provisional application No. 60/064,067, filed on Nov. 3, 1997.

(30) Foreign Application Priority Data
May 7, 1998 (US) PCT US98/09345

(51) Int. Cl. A61B 5/00

(52) U.S. Cl. 600/582; 600/365

(57) ABSTRACT

A non-invasive glucose monitoring device includes a mechanism for stimulating salivary glands secretion of saliva into oral fluid prior to collecting a sample of the oral fluid. A mechanism is provided for detecting the amount of glucose in the sample, a mechanism also being provided for quantitating blood glucose level based on the amount of glucose detected. A method of non-invasively monitoring glucose includes the steps of stimulating salivary glands secretion of saliva into oral fluid, collecting a sample of the oral fluid, detecting an amount of glucose in the sample, and then quantitating the blood glucose level based on the amount of glucose detected. These methods and devices are used to diagnosis individuals with glucose levels that indicate a diabetic disease state. The present invention is also usable to monitor the blood glucose levels of subjects who are being treated for diabetes.

### CROSS REFERENCE TO RELATED APPLICATIONS

This application is based on U.S. application Ser. No. 09/072,115, filed May 4, 1998, which is based on U.S. Provisional Application Ser. No. 60/064,067, filed Nov. 3, 1997.

### GOVERNMENT SUPPORT

The research carried out in connection with this invention was supported, in part, by a grant from the National Institutes of Health (IR43DK50500-01). The United States Government may have certain rights in the invention.

### BACKGROUND OF THE INVENTION

1. Field of the Invention

The present invention is directed to an apparatus and method for determining blood glucose content by the collection and analysis of oral fluid and further a method of diagnosing an individual suffering from diabetes based on the analysis of the oral fluid. More particularly, the present invention non-invasively collects oral fluid, oral fluid glucose content is determined, blood glucose levels are derived based upon the amount of glucose detected, and a diagnosis of diabetes is made based on blood glucose levels.

2. Description of the Related Art

The pathogenesis of diabetes originates in sustained or periodic elevations of blood glucose and glucose in tissues secondary to a deficiency in, or insensitivity to, insulin. Glucose is linked non-enzymatically to accessible reactive sites of proteins causing altered structure and function which leads in time to diseased organs. The grade of glycation depends upon glucose concentration and the amount of derivitized protein accumulated depends upon the lifetime of the individual proteins effected. Accordingly, the significance of maintaining reduced glucose concentrations is widely accepted.

Although early studies focused on Type I patients (Cohen, 1988), it is generally believed that Type II individuals and others not taking insulin would benefit from better diabetic control. Although many patients tolerate the pin prick necessary for the taking of an actual blood sample, followed by blood analysis, a bloodless, quick and convenient test using saliva can enlist Type II individuals into an effective, better diabetic control. Type I persons would also benefit to the extent that a bloodless test would reduce the number of finger sticks required. The existence of a convenient, non-invasive test can also permit prescreening of a large number of individuals using the newly promulgated 126 mg/dL criteria.

Many prior art patents discuss the analysis of glucose in various fluids, including saliva, but do not discuss the relationship of determining blood glucose from saliva levels nor do they discuss any specific devices for obtaining the same. For example, U.S. Pat. No. 3,947,328 to Friedenberg et al., issued Mar. 30, 1976, discloses a method, apparatus and test compositions for a rapid, accurate test of concentration levels of various components of body fluids, including glucose levels in saliva. An oxidizing test is utilized to determine the levels, but no relationship is disclosed relating glucose analysis in saliva to blood levels of glucose. U.S. Pat. No. 5,139,023 to Stanley et al., issued Aug. 18, 1992, discloses a method and apparatus for non-invasive blood glucose monitoring. Blood glucose is monitored non-invasively by correlation with the amount of glucose which permeates an epithelial membrane, such as skin or a mucosa membrane within the mouth. However, the Stanley patent specifically states that it is undesirable for such a sample to be contaminated by oral fluid, specifically saliva. Although the Stanley et al. patent discloses the step of taking a sample from inside the mouth, the sample taken is not a sample of oral fluid or saliva.

U.S. Pat. No. 5,056,521 to Parsons et al., issued Oct. 15, 1991, discloses an absorbent non-reactive collecting swab which is brought into contact with a favorable surface of the oral cavity. An interstitial transudate is selectively collected from the vestibule region of the oral cavity at the con unction of the superior labal mucous membrane and the superior gingivae between the upper canine teeth. The fluid collected is then squeezed out from the swab into a monitoring instrument located off site. The patent goes into great detail to note that, although general statements are made with regard to oral fluid, the system requires that the sample be taken from the specific mucous membrane described above so that the sample is devoid of uncontrolled oral fluid that might distort the glucose level in the sample by the diluting the desired fluid (namely, interstitial transudate, column 3, lines 35-40, of the Parsons et al. patent). From this sample, glucose levels of the sample itself are determined, the specification being devoid of any teaching of how blood levels of glucose can then be obtained. Hence, the Parsons et al. patent does not disclose any method or apparatus for utilizing whole oral fluid to determine blood glucose levels and, in fact, teaches away from using the same or from diluting a sample with such oral fluid.

Diabetes

Approximately 16 million Americans and 120 million people worldwide are estimated to have diabetes. Without proper management of the disease, diabetes leads to severe complications such as blindness, kidney disease, heart disease, nerve damage and death. Diabetics control their glucose levels through blood glucose monitoring to determine insulin injections and behavior modification. Although advised to test glucose levels 4 to 7 times a day, most diabetics take readings only 1 to 2 times a day or less due to the pain and inconvenience of obtaining a finger stick blood sample. Despite the need for non-invasive glucose monitoring systems, the glucose testing market is growing rapidly. Worldwide sales of products for blood glucose self-monitoring were approximately $2.5 billion in 1996, reflecting an increase of 14% over 1995.

Diabetes is a chronic disease characterized by the body's inability to produce or properly use insulin, a hormone that is needed to convert sugar, starches and other food into energy needed for daily life. Diabetes is classified by the presence or absence of insulin in the body and diabetics are generally classified into one of two major categories: Type 1 diabetics do not produce insulin due to pancreatic cell destruction, and Type 2 diabetics have resistance to insulin and/or an insulin secretion defect. Type 1 diabetics need insulin just to survive and this form is found most frequently in children and young adults. Approximately 40% of Type 2 diabetics require insulin injections. There are several forms of less common diabetes, which are usually associated with various medical conditions such as gestational diabetes mellitus (GDM) which is first diagnosed during pregnancy.

People demonstrating impaired glucose tolerance (IGT) are regarded as “borderline” diabetics. They have fasting blood glucose values that lie in the normal range, but the values are raised more than normal following ingestion of a measured glucose load. Many of these people go on to develop Type 2 diabetes, so it is important that they be clinically monitored on a regular basis.

Approximately 16 million Americans and as many as 120 million people worldwide are estimated to have diabetes. However, the American Diabetes Association (ADA) estimates that only half of the diabetics in the U.S. have been diagnosed. Type 1 diabetics make up 5-10% of the diabetic population, while the more common Type 2 diabetics make up the rest. The incidence of Type 2 diabetes is on the rise due to aging populations, changing diets, a greater prevalence of obesity and a sedentary lifestyle. The U.S. and Europe have traditionally been the most active in trying to identify and manage their diabetic populations. However, Asian countries such as Japan have recently announced new initiatives. Japan has an estimated 6 million diabetics of which only 1.7 million have been diagnosed.

Without proper management of the disease, diabetes leads to severe complications. Type 1 diabetics manufacture little or no insulin, so they depend on daily injections of the hormone to stay alive. This group must track blood glucose levels with vigilance in order to determine the correct dose of insulin. The much more common Type 2 diabetes progresses more slowly, and patients suffering from the illness typically do not seek medical care until they experience symptoms such as vision problems or numbness in the feet. Type 2 diabetes control their glucose levels through diet, exercise, drugs, and glucose monitoring. If left untreated, Type 2 diabetes can lead to blindness, kidney disease, heart disease, and nerve damage, potentially leading to amputation. Overall, the ADA estimates the direct and indirect cost of diabetes at over $92 billion per year in the U.S.

The criteria for diagnosis and classification of diabetes focuses on a patient's blood glucose levels and were revised by an ADA International Expert Committee in 1997. The ADA has recommended that all individuals worldwide above the age of 45 be screened every three years and that younger individual should be screened more frequently if they are in an at risk group. Also, in addition to existing category of “Impaired Glucose Tolerance,” the ADA has recommended the creation of a new state between “Normal” and “Diabetes” designated “Impaired Fasting Glucose.”

The ADA has also created the designation of diagnostic criteria for both non- fasting and fasting individuals for each category.

| | Casual | 2 Hour | 8 Hour |
|---|---|---|---|
| Category | (Non-Fasting) | Fasting | Fasting |
| Normal | <200 | n.a.^{f} | <110^{d} |
| Impaired Fasting | n.d.^{e} | n.a. | ≧110-<126 |
| Impaired Tolerance | n.d. | ≧140-<200 | n.a. |
| Diabetic^{a} | >200 | ≧200^{b} | ≧126 |

^{a}Any one of the three methods is considered diagnostic in itself by the ADA
^{b}By the 2 hour Oral Glucose Tolerance Test
^{c}A casual plasma glucose (taken at any time without regard to time of last meal). Must also present with the classic symptoms of increased urination, increased thirst and unexplained weight loss.
^{d}Upper lever of normal
^{e}Not determined
^{f}Not applicable

Currently, screening for diabetes is usually performed at the direction of a physician and involves either a venous blood draw for glucose measurement at a central 20 lab or by capillary blood (finger stick) for measurement by a point of care device in the doctor's office.

Doctors have long recommended that newly diagnosed diabetics measure their blood glucose when they get up in the morning, before and after every meal and before going to sleep. Since the publication of a 1993 landmark report by the American Diabetes Association that demonstrated tight blood glucose control leads to a reduction in diabetic complications, this recommendation has been extended to all diabetic patients.

To measure his glucose, a diabetic must disinfect his finger with an alcohol wipe. He then stabs his finger with a lancet, massages the finger to draw an adequate amount of blood, places a drop of blood on a glucose reagent strip, and then insert the strip into an instrument which provides a quantitative glucose reading. Although they are advised to test their glucose levels 4 to 7 times a day, on average most diabetics only take readings 1 to 2 times a day, primarily due to the pain and inconvenience of the current technology. In fact, according to the ADA, only 10-20% of Type 1 diabetics follow the recommended monitoring regimen of at least four tests per day and 21% don't monitor their glucose at all.

In addition to discomfort, cost is a major limiting factor in the expansion of glucose monitoring. While the current crop of glucose meters themselves are not expensive (manufacturer rebates can drop the final cost of the meter to $10 or less), the single use disposable strips cost from $0.50 to $1.00 a piece. Thus, a diligent tester could spend over $150 a month on strips and another $5 to $10 a month for lancets.

It has been said that no aspect of blood glucose monitoring is so distasteful to the diabetic as the prospect of pricking their fingers two to five times a day to obtain a blood sample. Most patients would welcome an inexpensive, convenient, and relatively non- invasive method to measure glucose and to diagnosis diabetes.

In view of the above, it would be desirable to develop a non-invasive means for determining blood glucose levels. It is also desirable to provide a simple means for doing so which does not require exclusion of oral fluid from a bucual cavity device.

### SUMMARY OF THE INVENTION

In accordance with the present invention, there is provided a non-invasive glucose monitoring device including stimulation means for stimulating salivary gland secretion of saliva into oral fluid and collection means for collecting a sample of the oral fluid. Detection means, operatively connected to the collection means, detects an amount of glucose in the sample and quantitation. means operatively connected to the detection means quantitates blood glucose levels based on the amount of the glucose detected.

The present invention also provides a method of monitoring blood glucose by stimulating salivary gland secretions of saliva into oral fluid, collecting a sample of the oral fluid, detecting an amount of glucose in the sample, and finally quantitating blood glucose level based on the amount of glucose detected.

One embodiment of the present invention is directed to a noninvasive method of diagnosing diabetes comprising, stimulating salivary gland secretion of saliva into oral fluid, measuring saliva glucose levels in a subject, and diagnosing a diabetes disease state in said subject.

In one aspect of this embodiment, the measuring step further comprises, collecting a sample of oral fluid, detecting an amount of glucose in the sample, detecting an amount of glucose in the sample, and quantitating a blood glucose level based on the amount of glucose detected. In another aspect of this embodiment, the measuring step comprises providing said subject a device for obtaining and measuring glucose levels in a saliva sample.

The device comprises a stimulation means for stimulating salivary gland secretion of saliva into oral fluid, a collection means for collecting a sample of the oral fluid, a detection means operatively connected to said collection means for detecting an amount of glucose in the sample, and quantitation means operatively connected to said detection means for quantitating blood glucose level based on the amount of glucose detected. In another aspect of this embodiment, the device further comprises a housing defining said collection means, the housing containing said stimulation means for release into a buccal cavity. Another aspect of the invention relates to the diagnosing step of the present method. The diagnosing step comprises reading a glucose level obtained from said subject and comparing said glucose level to a range of results, whereby said diagnosis of diabetes is made based upon said glucose level.

Another embodiment of the present invention encompasses a noninvasive method of monitoring glucose levels comprising providing a subject being treated for diabetes, stimulating salivary gland secretion of saliva into oral fluid, measuring a saliva glucose level from said patient, and determining a treatment course of action based on said saliva glucose level.

One aspect of this embodiment addresses the measuring step of the present invention's method. In this aspect, the measuring step comprises providing said subject a device for collecting a sample of oral fluid, detecting an amount of glucose in the sample, and quantitating a blood glucose level based on the amount of glucose detected.

In another embodiment of the present invention, the device comprises a stimulation means for stimulating salivary gland secretion of saliva into oral fluid, a collection means for collecting a sample of the oral fluid, a detection means operatively connected to said collection means for detecting an amount of glucose in the sample, and quantitation means operatively connected to said detection means for quantitating blood glucose level based on the amount of glucose detected.

Still another embodiment of the present invention is a method of monitoring blood glucose by stimulating salivary gland secretion of saliva into oral fluid, collecting a sample of the oral fluid, detecting an amount of glucose in the sample, quantitating a blood glucose level based on the amount of glucose detected, and determining a treatment action in view of the blood glucose level quantitated.

### BRIEF DESCRIPTION OF THE DRAWINGS

Other advantages of the present invention will be readily appreciated the same becomes better understood by reference to the following detailed description when considered in connection with the accompanying drawings wherein:

FIG. 1 is a perspective view of an oral fluid collection device in accordance with the invention;

FIG. 2 is a cross-sectional view based substantially along lines 2-2 of FIG. 1;

FIG. 3 is a perspective view of a second embodiment of the invention;

FIG. 4 is a perspective view of a third embodiment of the invention;

FIG. 5 is a schematic plan view of a fourth embodiment of the present invention;

FIGS. 6A-B are graphs showing glucose standard curves in buffer or saliva indicating a comparison of selected chromogens wherein FIG. 6A shows spiked buffers and saliva and FIG. 6B shows only spiked saliva;

FIG. 7A-B are graphs illustrating a glucose standard curve wherein

FIG. 7A is a standard curve for in phosphate buffer and

FIG. 7B is a standard curve and assay variation;

FIG. 8 is a graph showing the time to saliva glucose equilibrium in the subject invention;

FIG. 9 is a graph showing the effect of pH on the glucose assay;

FIG. 10A-B are graphs showing oral glucose contamination of saliva following ingestion, wherein

FIG. 10A shows oral glucose ingestion being present and

FIG. 10B are results where there was no ingestion of glucose;

FIG. 11A-C are graphs showing glucose collected by the present invention compared to finger stick glucose (A and Q and venipuncture (C) in hyperglycemic and normal subjects, FIG. 11A showing the results of 13 diabetic subjects, FIG. 11B showing a collection of data from subjects from the present study and an earlier study as described in the specification; FIG. 11C showing glucose collected by venipuncture vs. SalivaSac™ glucose, and FIG. 12A-B are graphs showing a correspondence between saliva glucose and venipuncture blood, FIGS. 12A showing venipuncture vs. stimulated subject using the SalivaSac™ (present invention) for collection of saliva, and FIG. 12B shows the same comparison but using all subjects, not only stimulated subjects using the SalivaSac@.

FIG. 13 is an exploded depiction of an electrochemical strip containing saliva sampling device.

FIG. 14 is a graphical representation of the steps involved in monitoring glucose levels in a subject.

FIG. 15 is a graphical representation of blood glucose measurements taken from diabetic and normal patients.

FIG. 16 is a graphical representation of glucose measurements taken from diabetic and normal patients.

FIG. 17 is a graphical representation of ROC curves correlating sensitivity and specificity for the saliva glucose test of the present invention.

### DETAILED DESCRIPTION OF THE PREFERRED EMBODIMENT

The present invention provides a non-invasive glucose monitoring device and method, the device including a mechanism for stimulating salivary gland secretion of saliva into oral fluid, a collection apparatus for collecting a sample of the oral fluid, a detection mechanism operatively connected to the collection device for detecting an amount of glucose in the sample, and a quantitation mechanism operatively connected to the detection mechanism for quantitating blood glucose level based on the amount of glucose detected. Thus, the elements of the present invention most generally are (1) simulation of salivation; (2) insertion of a collection device into the mouth for the period of time required for the contents to reach equilibrium with whole saliva; (3) withdrawal from the mouth of the collection device and transfer of the sample to a detection mechanism, such as a qualitative test strip as discussed below in which glucose concentration is estimated; and (4) means for calculation of estimated blood glucose. Such a system can be a integrated device wherein stimulation, collection, and quantitation are accomplished on a single strip or can be a non-integrated device, for disposal, in or out of the mouth, as discussed in greater detail below. The device is non-invasive, so it removes resistance to testing and can be used in public. It can be made inexpensively, thereby lowering economic barriers to benefits of the device. It can be a single use device and thereby avoid the spread of an infection and is also easily transportable. It is also a simple device thereby requiring little to no training for its use. Hence, the present invention, as most broadly defined, provides significant improvements over the prior art.

More specifically, the term “oral fluid” is not simply saliva, but rather the liquid contents of the mouth which include cellular secretions, components from food, saliva, as well as other components which may be secreted into the mouth, regurgitated into the mouth, or brought into the mouth by airborne means.

Oral fluid has a glucose concentration that has approximately 1/200 to 1/100 of the contemporaneous blood concentration. Accordingly, measurement of oral glucose can be used to estimate blood glucose.

Prior to the development of the present invention, there were few reports in the literature concluding that a general correspondence between concentration of blood and saliva glucose or whole oral fluid glucose exists. As stated above, many prior art devices excluded saliva and other oral fluid, maintaining that the inclusion of such would cause inaccuracies in glucose measurements. Borg and Berkhed (1988) demonstrated the correlation following oral loading with 75 grams of glucose. In accordance with the present invention, it is proved that Borg and Berkhed were measuring an artifact in which contamination of oral mucosa in the interval following ingestion of glucose falsely mirrored the rise in blood; Reuterving et al. (1987) measured glucose secretions of three individual salivary glands and showed that the closest correspondence with blood is in fluid from the parotid. These investigators also claimed the existence of a threshold for the spill over of the plasma glucose into the saliva of 10-15 mmL/L (180-260 mg/dL). A threshold of this type is analogous to well characterized glucose threshold of renal tubules. If this threshold identified by Reuterving et al. is accurate, then saliva cannot be used to detect glucose below about 200 mg/dL.

The data disclosed in the example section below shows that if a threshold exists, it must occur at blood concentrations substantially less than 200 mg/dL. The problem with the published work cited above is that the investigators used a standard Trinder assay, and the analytical variations seen in whole saliva, particularly at the lowest concentrations, render conclusions on detection of “zero” saliva glucose highly suspect. It is concluded based upon the present work that a new, more sensitive glucose oxidase-peroxidise chemistry in combination with the present invention makes it possible to follow saliva glucose concentrations to the lower concentrations secreted as blood declined to hypoglycemic levels. The results set forth herein show a threshold for saliva glucose to exist at least as low as 70-100 mg/dL, depending on the subject, approximately at least one half of the blood concentration specified by Reuterving et al. Based on the above, the present invention is at least useful as a diagnostic for elevated blood glucose and can certainly be predicted to be useful for lower blood glucose as well.

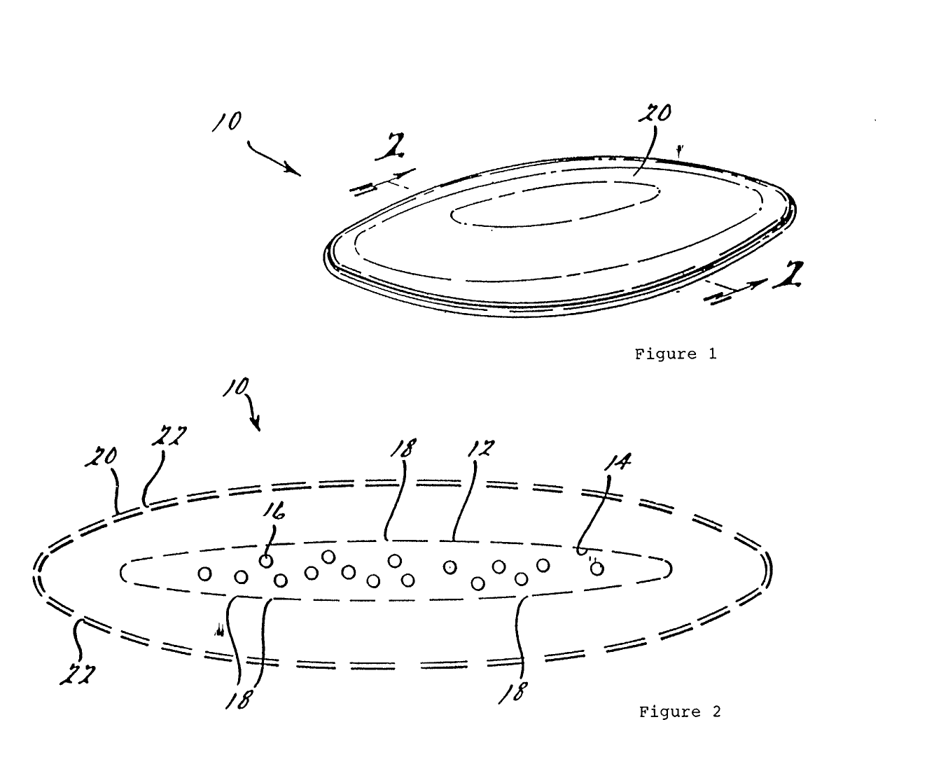

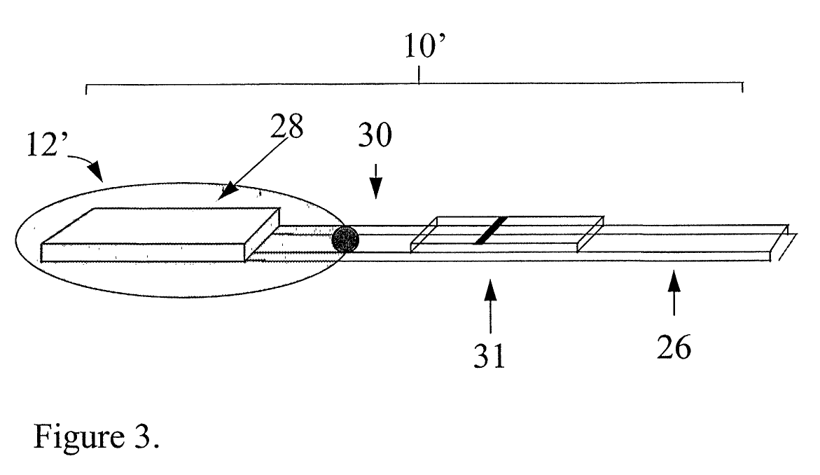

The collection device, generally shown at 10 in FIG. 1 and 2 is preferably an oral fluid collection article disclosed in detail in U.S. Pat. No. 4,817,632 to Schramm, issued Apr. 4, 1989, and assigned to the assignee of the present invention. The collection device is generally an ovoid small disc or pillow-shaped article adapted to fit in the mouth of a patient. The article includes a semi-permeable membrane 12 which defines an enclosed chamber 14. The chamber can include an osmotic substance 16 which is totally enclosed by the semi-permeable membrane 12.

The semi-permeable membrane 12 is made of a substance which has a plurality of pores which are of a suitable size to allow for the collection of oral fluid or which acts as a filter for filtering out unwanted particulate matter or larger molecules such as binding proteins from the sample. An example of such a membrane is Cuprophan@ manufactured by Enka AG, a division of Akzo, Inc. This membrane is available as flat sheets or in a tubular form, both of which can be cut to the appropriate size. The membrane is composed of regenerated cellulose and has a nominal molecular weight cut-off of 12,000 daltons. The molecular weight cut-off, also termed the exclusion limit, is central to the function of the semi-permeable membrane. The pore size of the membrane is such that molecules larger than 12,000 daltons; (such as proteins, polysaccharides and particulate matter) cannot cross the membrane 12 to enter the central compartment 14. In this way, the fluid obtained by the collection device is filtered saliva (more specifically, ultrafiltered saliva), a uniform non-viscous sample required for accurate measurement of glucose (molecular weight, 180 daltons). Any membrane, filter, fabric, paper, mineral, plastic or other material capable of allowing the passage of glucose while excluding the viscous, particulate or cellular material of oral fluid, could be used in the collection of filtered saliva. Other dialysis membranes having a range or exclusion limits could also be used, provided such membranes are permeable to glucose and allow its transport from whole saliva to the central compartment.

The osmotic substance 16 is soluble in oral fluid thereby providing an osmotic pressure inside the chamber 14 for drawing oral fluid from the mucosal cavity of the patient into the chamber 14. The membrane 12 retains at least a portion of the oral fluid in the chamber 14 for later removal, as discussed below. The osmotic substance can be a crystalline or an amorphous material which is soluble in saliva and allows interference-free analysis of the sample for whatever particular analysis is being undertaken to determine the glucose levels. Alternatively, the osmotic substance can comprise a high molarity solution of a crystalline or amorphous material which is dissolved in water or some other non-interfering solute. The osmotic substance must be non-toxic in nature and is preferably palatable.

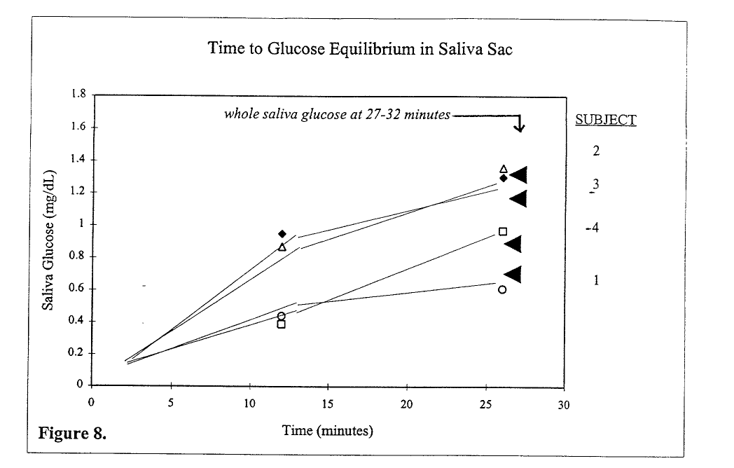

The osmotic substance can also take the form of a stimulant of salivation. For example, the osmotic substance can be selected from the group including salts, sugars, amino acids, other organic acids and small peptides. The preferable osmotic substance is one which dissolves readily when hydrated by the moisture in oral fluid, establishes, when dissolved, an osmotic pressure capable of drawing additional fluid across the filtering surface, and is compatible with subsequent measurement of glucose in the sample obtained. In example, the osmotic substance used in collection of samples forming of the data presented in FIGS. 7-12 is sodium citrate. This salt also has the effect of stimulating salivation, the first element of the present invention. A mixture of salts or other substances can also be used. An example is sodium citrate mixed with a small amount of citric acid, the latter acting to further stimulate salivation.

The basic elements of the present invention are retained if a non-osmotic material is used to collect filtered saliva. For example, absorbents or adsorbents can be used to collect saliva if they provide a method for the separation of glucose from the viscous large molecular-weight materials of whole saliva. Completely different physical forces and methods could also be used to obtain a filtered sample of oral fluid. For example, a vacuum could be created to draw oral liquid by aspiration through a filtering surface with deposition of the glucose-containing fluid in a sink. Or a positive pressure could be exerted on a saliva sample, forcing liquid through a rigid filtering surface with elaboration of filtered liquid into a central or lower compartment. One example would be a conventional filtration tube in which whole saliva is forced from an upper to a lower camber by positive pressure or by centrifugation, or by application of a negative pressure or vacuum to the lower chamber. Though the preferred embodiments illustrated in FIG. 1-5 are based on the patent SalivaSac@ with its features which allow direct insertion into the mouth, the claims of the present invention are also extended to any in-the-mouth or external device capable of producing a filtered sample of oral fluid containing a concentration of glucose equivalent to that in whole oral fluid. The expanded claims embody specifically any device or method in which expectorated saliva or oral fluid is processed further by a device external to the oral cavity which obtains an accurate measure of glucose.

Stimulation of salivation has been found to be critical. Preliminary data set forth herein is indicative that much of the controversy surrounding the correspondence between blood and saliva glucose or full oral fluid glucose can be traced to analytical imprecision associated with the sticky, viscous, and generally variable qualities of whole saliva or whole oral fluid. Testing in a limited number of subjects indicated that the blood-saliva relationship was improved by the use of the ultrafiltrate obtained by the collection device after citric acid stimulation was made in accordance with the present invention. It was felt necessary to show that the contents of the collection device accurately reflect whole saliva glucose concentration since it is whole saliva that is derived in the first instance from blood; blood glucose enters the primary secretion of salivary glands principally by paracellular diffusion through leaky epithelial cellular junctions. The rate of diffusion (and thus the amount of glucose transported per unit of time) will be increased as blood glucose rises. A minor pathway is transcellular mediated by the apocerine secretion of glandular cells (Baum, 1993). Accurate measurement of glucose in whole saliva is possible provided numerous processing steps are first employed to produce the equivalent of a filtered sample. Thus, glucose concentration in whole saliva was determined after: (1) sonication of sample at 1600 Hz (hertz); (2) freezing and thawing the sample to precipitate large molecular weight interferences; (3) centrifugation at 3 000×g for 10 minutes; (4) heating the sample to 100° C.×10 minutes to eliminate glucose- and carbohydrate-hydrolyzing activities (enzymes); (5) adjusting pH to optimal assay pH (pH 6.5-7.5). This procedure produced accurate measurement of glucose to 0.06 mg/dL, as shown by quantitative recovery of glucose spiked into such samples. (It can be noted that the filtration properties of the preferred embodiment of the present invention produce a sample that is equivalent to the five-step processed whole saliva described immediately above).

The ability to measure glucose in saliva allowed for the reexamination of the time necessary for the container made in accordance with the present invention to reach equilibrium with whole saliva glucose. Subjects were observed and were found to be variable in the time to reach equilibrium, but it could require as much as 20 minutes. The more viscous and protein that enriched the saliva, the longer the time needed to reach equilibrium. Individuals with copious salvia, clear in appearance and relatively impoverished in protein often reached equilibrium at approximately the time the last of the crystalline osmotic driver in the container dissolved, six to seven minutes.

As a consequence of these results, the desirability of a dilute saliva by stimulation of salivation was recognized. The desirability of scaling down to a smaller size of the collection device was also recognized. A small device will reduce diffusion distance and destination volume and will also increase surface to volume ratio. These three factors are the principal determinants of the time required to reach equilibrium with surrounding whole oral fluid.

It was further found, as is demonstrated in the Examples below, that stimulated saliva glucose more closely parallels blood glucose then did unstimulated saliva. This was a critical discovery. It was also found that stimulation of saliva secretion also reduced protein content of saliva and elevated sodium concentration while having a modest effect on potassium.

The conclusion from the physiological finding above is that stimulation forces saliva quickly through salivary ducts and this minimizes reabsorption of glucose, water, and sodium ions by salivary gland ductal transport systems. Therefore, stimulated saliva more nearly reflects the composition of the primary filtrate-secretion elaborated by the secretory portion of the salivary glands and it is this fluid that is derived by passive diffusion from blood. Accordingly, as described above, it is preferred to provide a stimulatory component. This is preferably accomplished, as stated above, by stimulatory component being disposed within the container 10 for release therefrom. As also stated above, the preferred stimulant is citric acid.

Referring again to FIG. 2, the semi-permeable membrane 12 may be enclosed by an outer protective membrane 20 which includes macroscopic pores and is disposed about and completely exposes the membrane 12. The outer protective membrane 20 can be made of any material which would be generally pliable, tasteless, and non-toxic. Preferably, silicon materials or other materials are selected which have substantial mechanical strength to protect the inner membrane from damage due to biting by a patient and similar hazards which may be associated with the use of the present invention in a patient's mouth. The outer membrane can be made from many materials whereby saliva can pass through easily, the material having microscopic pores 22. Alternatively, the present invention can include a container 10 as described above without the use of the outer membrane 20, wherein the inner membrane 12 is made of material of sufficient mechanical strength to survive in the environment of the mouth of a patient.

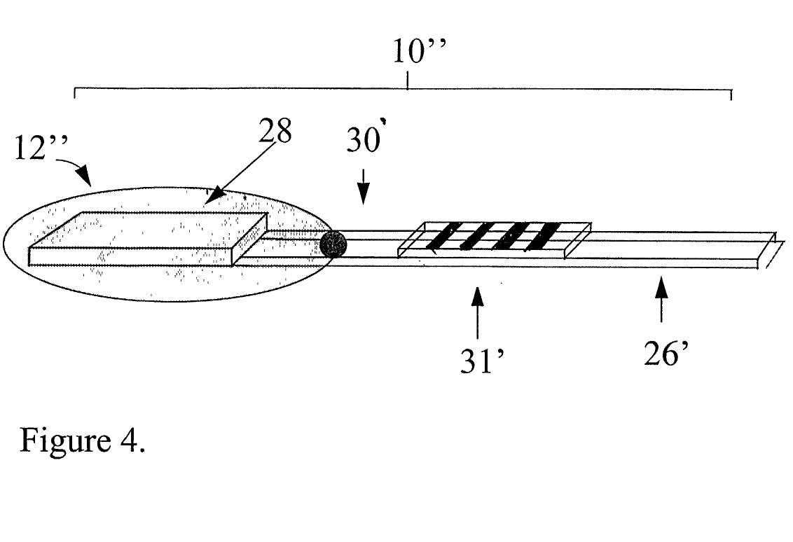

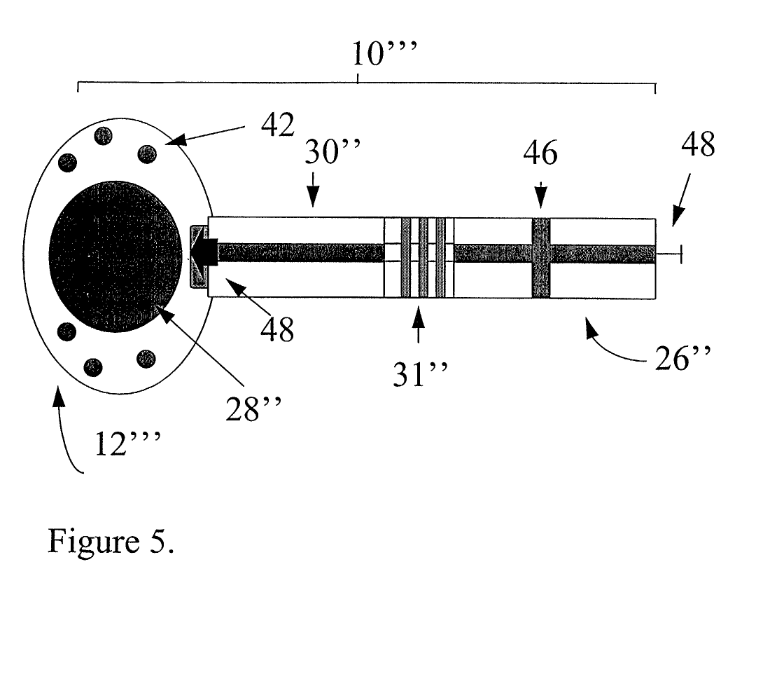

A preferred embodiment of the subject invention is shown in FIGS. 3 and 4. A device 24 is in the form of a test strip including a support 26. A membrane sac 10′, having a structure as described above, is mounted over one end of the strip 26 and contains an absorptive matrix 28. Absorptive matrix 28 can include the stimulator of salivary gland secretion, such as sodium citrate. The absorbent matrix 28 is in fluid communication by abutment with a threshold-type indicator film 30.

The film contains the enzymes glucose oxidase and horseradish peroxidase (or some other peroxidase) and a combination of dyes and accessory reagents, such as buffers and stabilizers, which are capable of producing a colored spot or line in which color intensity is proportional to the amount of glucose in the sample. Glucose oxidase applied as a dry reagent to the strip hydrolyzes sample glucose to gluconic acid with production of hydrogen peroxide The peroxidase converts the peroxide product to water and uses the electrons produced to react with the dyes to form a colored compound. The color intensity, as noted, is scaled to the amount of glucose initially present in the sample. Nuanerous enzyme-based glucose-sensitive strips of the general type described exist. Various dyes have been used to generate the final color product. Some of these are described in the Examples herein.

The present invention includes any type of solid-phase strip chemistry capable of determining glucose at the concentrations existing in filtered saliva or oral fluid. Moreover, as the essential elements of this invention are the use of a filtered and stimulated saliva, any method of glucose measurement could be associated with the processed sample. These include, but are not limited to, other enzyme-based system (e.g., using hexokinase or glucose dehydrogenase or any glucose metabolizing enzyme), chemistry-based systems (e.g., a specific glucose reagent producing some quantifiable signal), and glucose sensors (e.g., glucose-specific electrochemistry).

Utilizing this embodiment of the invention, oral fluid is collected within the container 10′ by the absorbent matrix 28. Upon contact with the oral fluid, sodium citrate is dissolved and released through the container 10′ thereby stimulating saliva secretion. The collected oral fluid is retained in the central compartment 28 for the period required for contents to reach equilibrium with whole oral fluid glucose. In a small collection device, this time may be two or fewer minutes. The contents of the sac are then exposed to one end of the colorimetric glucose strip. The mechanism retaining of the filtered liquid can be a simple pressure-sensitive opening (port), or the rate-of-flow of sample along the test strip can be made sufficiently slow to ensure that sample has reached glucose equilibrium.

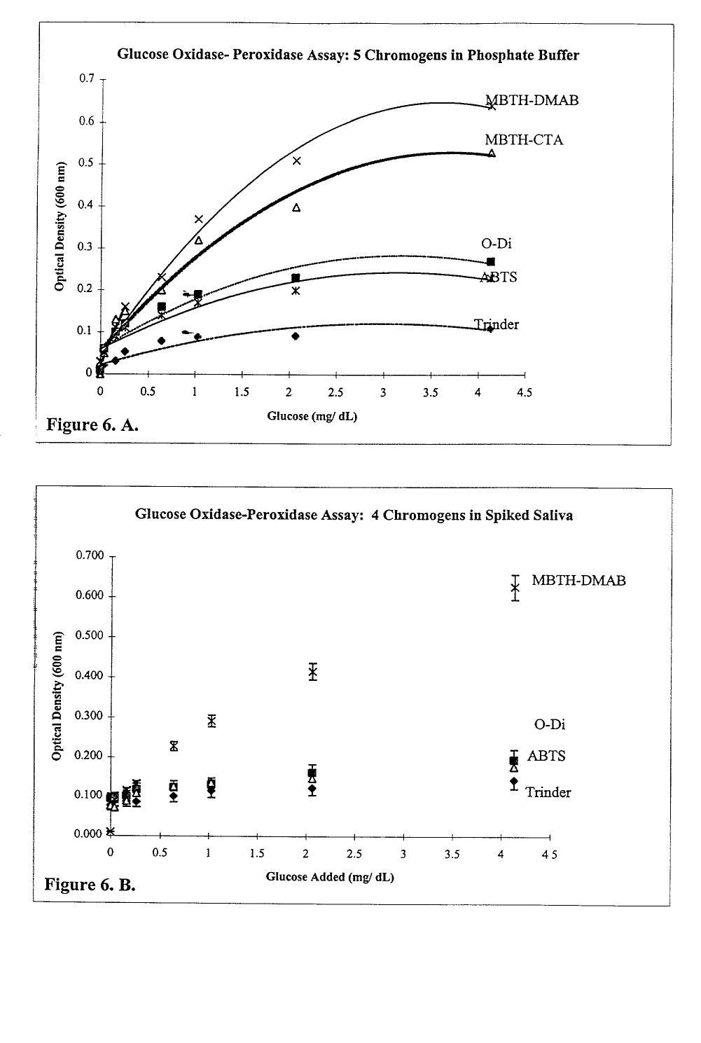

An alternative embodiment of the present invention is generally shown at 32 in FIG. 4. A support strip 26′, which can be similar to that shown in FIG. 3 supports a collection container 10″ as described below. The collection container, containing the absorbent matrix 28′ which can also contain the sodium citrate, is mounted adjacent a wicking material 34 in communication with a thermometer-type indicator film 36. A thermometer type film is one in which the enzymes and dyes required to produce the calorimetric signal are arrayed from proximal to distal on the test section of the strip. As sample moves through the test zone, glucose is depleted and colored products are formed. When glucose is exhausted from the sample, no further color development can occur in the distal enzyme field. The amount of glucose in the sample is thus proportional to the linear distance of color development. A thermometer-type strip requires that an accurately measured fixed volume of sample be applied to the strip. This can be achieved in this embodiment by creation of a saturable strip having a limited (and fixed) capacity for liquid absorption, by timing the reaction to allow a known volume of sample to enter the test zone, or by application of a known sample volume obtained by a chamber of defined volume between sample and strip. The flow of liquid and its glucose up the strip proceeds by capillarity according to well known principles. The strip may contain accessory elements, such as sample volume adequacy indicators, as shown in FIG. 5, additional filtration materials, and test sections to check quality of reagents.

The indicator film can be graded to provide an indication of blood glucose level correlated from the glucose content of the collected oral fluid. Thus, the film 36 provides a detection mechanism, as well as a quantitation mechanism. Alternatively, a container 10, 10′ or 10″, mounted on a strip 26, 26′ or independent thereof shown in FIGS. 1 and 2, can be transferred to a detection device known in the art for glucose analysis. A glucose level can then be correlated to blood glucose levels.

One such embodiment would require placing the strip into a reflectance spectrophotometer similar to those currently used in monitoring blood glucose. The strip could be moved to the monitor after the sample is introduced onto the strip, or a small integrated monitor could be created to present a combined replaceable strip-plus- collection device into the mouth or sample receptacle (for the embodiment using a device external to the mouth to process the saliva sample).

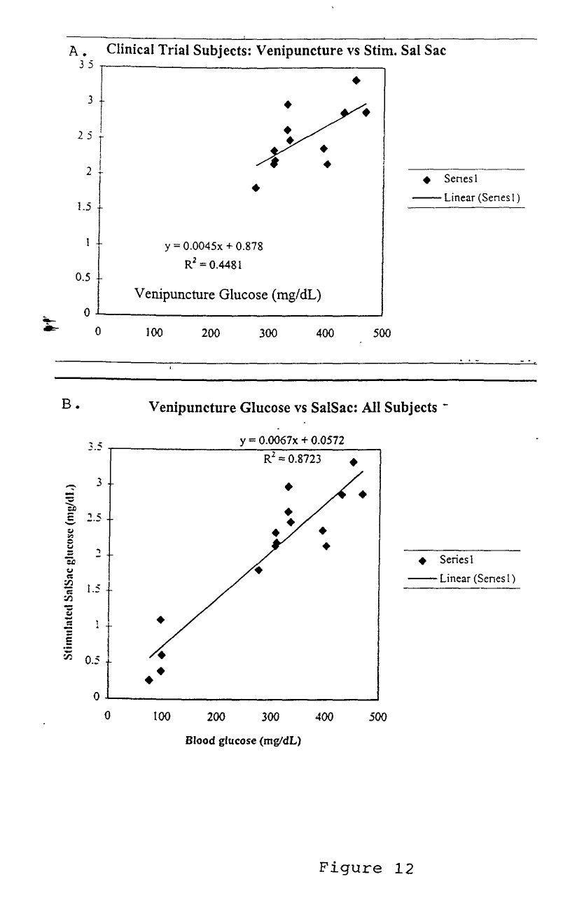

The correlation with blood is obtained by solving an equation which relates blood glucose to oral fluid glucose concentration. For example, solving the linear equations shown in FIGS. 11 and 12 for “x”, will produce the blood glucose concentration when the oral fluid glucose (y) is known. The exact quantitative values of the constants in this equation have not yet been determined. The nature of these constants could take one of two forms: (1) if most individuals show the same saliva to blood glucose ratios, a single equation can be developed for the subject populations; (2) or if individuals show different ratios, then each individual will be required to calibrate the saliva test against periodic measurements of their own blood glucose. In each situation, a simple equation is produced. It is understood that in actual use, the solution to the equation may be translated into an easily readable table or color chart. In the embodiment in which the collection device and strip test are incorporated into a reflectance spectrophotometer, the computation of the blood glucose concentration can be achieved by insertion of a dedicated computational chip into the monitor. These electronics thus convert a spectrophotometric signal into an estimated blood glucose value.

The preferred embodiment of the present invention is shown schematically in FIG. 5. Again, the container 10 . . . includes an osmotic component 40 contained within an inner membrane 12′ and a citric acid component 42. The container 10 . . . is mounted at the end of a wicking material 44 supporting a plunger 46 containing a needle 48 therein. The needle can be used to puncture the outer and inner membranes 12′, 42 to release the collected oral fluid therefrom onto the wicking material 44. The fluid wicks across the material 44 to the indicator portion 46. This embodiment allows for a retention of the sample in the central compartment until the user elects to admit the sample to the strip. Thus, the voluntary act of breaking a seal or barrier is required. This embodiment would be used if it takes an unusually long time for the collection device to reach glucose equilibrium in some subjects, or if the subject prefers to analyze the sample at a later time. Therefore, the device contains a membrane-osmotic driver collection component, a dispenser of citric acid, a mount for attachment of the disposable cross-strip, and a mechanism in the form of a pin or, alternatively, a pressure-sensitive valve, to penetrate or open the container to allow a measured volume of sample of oral fluid to be transferred to the test strip. Alternatively, as shown in the various embodiments, wicking materials can be used as a means for transferring an adequate sample as indicated by an adjacent indicator on the strip.

Diabetes Diagnosis and Monitoring

To be accepted by the diabetic and medical community, a non-invasive glucose testing system must adequately address the criteria of cost, speed, accuracy, ease of use, and portability. The system must be affordable as compared to current technologies. Results from the device should be available to the user in under 3 minutes (including device “set-up” time). Although the accuracy of home detection kits may vary, it is generally accepted that any home blood glucose test system should show a “real world” correlation coefficient of at least 0.85 to venous blood glucose. Finally, since diabetes afflicts people of all ages and socio-economic levels, any non-invasive glucose system developed must be easy to use for all.

The devices of the present invention are designed to overcome the disadvantages of using saliva as a diagnostic fluid. Embodiments of the present invention are collectively referred to as SalivaSac® collection devices, or more simply, a “sac”. The sac is specially designed to collect an ultrafiltrate of saliva directly in the buccal cavity. An osmotically active substance such as a salt is enclosed in a pouch consisting of a semipermeable membrane to form a disc of about 35 mm or less in diameter. The membrane consists of either cellulose or a synthetic copolymer. The device is taken into the mouth by the patient and sucked on.

Previous research has shown that an accurate measure of saliva glucose requires use of the Sac and stimulation of salivation. That is, unstimulated whole saliva yields highly variable results due to its content of sugar-producing and hydrolyzing enzymes. One embodiment of the Sac is an 11 mm (diameter) circular sac composed of an envelope of dialysis membrane filled with an osmotic drive, such as 10 mg of Na₃Citrate.

As discussed above, a feature of the present invention is the method of obtaining a saliva sample from a subject. In one embodiment of the present invention, an osmotic driver is used to promote saliva production. For example, when placed in the mouth, a citrate “osmotic driver” dissolves immediately, establishes an osmotic gradient across the membranes, and collects a sample of filtered saliva. This ultrafiltrate sample consists of water and those saliva analytes capable of convection or diffusion through the membrane. By eliminating the viscous components of saliva, and also excluding salivary enzymes, the sample can be measured accurately for glucose concentration.

The current version of the Sac requires a period of time in the mouth for the sample to attain the volume necessary to measure glucose. This time period can range from less than one minute to greater than thirty minutes. Salivation is stimulated in response to the application of the osmotic driver under the tongue. Work to date has shown that after stimulation, Sac glucose more closely mirrors the blood concentration than unstimulated saliva. Improved correspondence is obtained from the more rapid flow of stimulated saliva from glands through the salivary ducts. At higher velocity, less glucose can be removed by ductal reabsorption, and liquid discharged into the oral cavity more closely duplicates the initial equilibrium concentration of glucose in the primary filtrate formed at the interface between epithelial cells and plasma.

As discussed above, the presence of the SalivaSac in the mouth moderately stimulates salivary flow, and the sac can be lightly flavored with citric acid to further stimulate salivation. While the sac is in the mouth, saliva begins to enter through the semipermeable membrane which causes the salt or other osmotic driver in the SalivaSac to dissolve. The dissolved contents are initially at a very high concentration, thus creating an osmotic pump which draws saliva rapidly through the membrane until the sac fills, usually within 1 to 2 minutes depending on the pore size of the membrane and the sac capacity. The filled contents of the sac is an ultrafiltrate of saliva, and is clear, clean and potentially sterile. This fluid is ready to use directly, without further processing, as a diagnostic medium. For most analytes, it does not even need to be refrigerated. Depending on the analyte of interest, it is possible to vary the pore size of the membrane and the substance used to fill the sac in order to maximize the efficiency of collecting the desired analyte. In addition, depending on the amount of saliva required, the sac can be made in different sizes and formats, (e.g. tethered, attached to a plastic handle, directly linked to a lateral flow assay, etc.).

One embodiment of the present invention is a glucose level screening test. This test is used to identify individuals who may be suffering from diabetes. In this embodiment, the screen device can be used on subjects who have fasted in a casual manner before testing, or those subjects who have fasted. In subjects who have fasted casually (e.g., 2 hour Fasting) test results indicating a glucose level of <200 mg/dL are presumptively negative. However, for subjects who give a reading of >140 but <200, the test should be repeated. Subjects who show a reading of >200 are presumptive positive for diabetes and should consult a physician immediately.

In this embodiment, glucose levels of a subject are determined by examining to saliva of the tested subject. In one aspect of this embodiment a visual indicator of glucose level is utilized. In another aspect, a visual colorimetric chromatographic semi-quantitative glucose assay will be used.

When used to test a subject who has fasted, for example overnight or for an eight hour period, the relevant concentrations of glucose differ. Subjects tested with glucose levels of <100 are considered normal. Subjects who show levels of 100 to 100-126 are considered impaired, under the ADA system. For these subjects the test should be repeated to confirm the results and/or the subject shouls see a physician immediately. Subjects who give readings of >126 are presumptively positive and should seek the care of a physician immediately.

In an embodiment of the present invention design for diagnostic purpose comprises a simple disposable unit with a plastic housing that also comprises a visual calorimetric assay capability. The unitized saliva collection and testing device collects saliva at one end of the disposable by placement in the mouth for a sufficient period of time to gather a sample. Salivation is stimulated with citric acid and an osmotic driver, such as sodium citrate, facilitating the instantaneous importation of ultrafiltered saliva into the sac.

The interior of the sac will be in contact with the distal end of the chromatographic strip which will upon wicking (an additional 1-2 minutes flow time) produce a calorimetric response on the strip.

One suitable colorimetric chemistry system is available from Actimed, Inc. (Brunswick, N.J.). This system yields a semi-quantitative visual result. The visual response of the strip, housed in a clear plastic device, yields a thermometer-like response. The outside of the device will clearly indicate the relative concentration of glucose according to a scale printed directly on the plastic housing. The patient will read the level relative to the scale.

The subject using the above described embodiment will observing the reading produced by the device and determine a value that corresponds to the glucose concentration present in the subject's saliva. The subject will then compare the reading taken to the package insert and the directions for use present thereon. The directions will indicate that for non-fasting patients, a product response (suspect diabetic -see your physician) will be at the saliva equivalent of 200 mg/dL blood equivalents. For a fasting individual (>8 hours)—either a positive (>126 mg/dL blood equivalent; see your physician); an impaired (>110 to <126); or a negative (<110 mg/dL blood equivalent) response will be obtained using the appropriate scale. Depending upon assay reproducibility and other inherent product limitations, the response obtained with the screening product will be expressed either as normal, impaired or suspect positive (or alternatively as actual mg/dL blood equivalents).

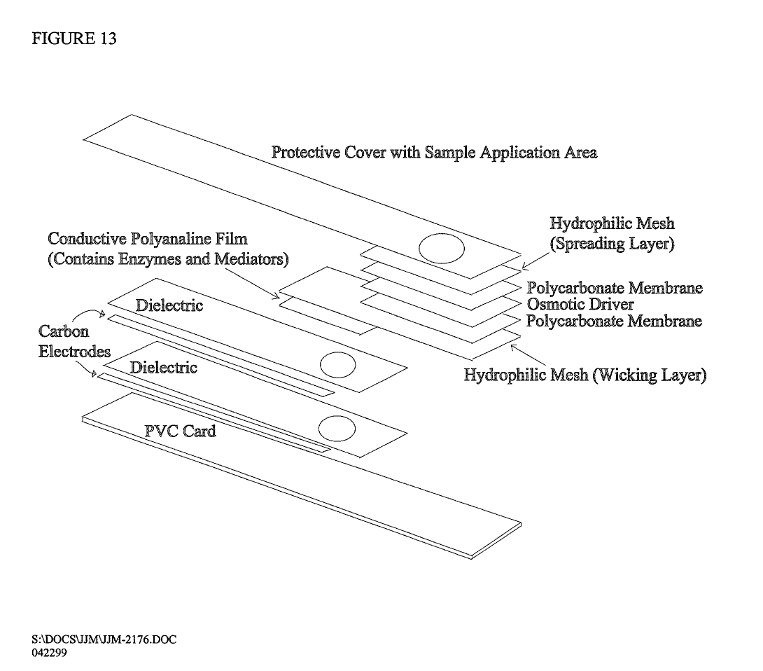

Another embodiment of the present invention is directed to a method of using the devices of the present invention in a diagnostic and/or monitoring test. For example, this embodiment has utility in monitoring Type 1 diabetics and Type 2 diabetics taking insulin and/or wanting better control of their glucose levels. This embodiment can employ an electrochemical sensor to determine the level of glucose in the sample. For example, an instrument-based testing method employing a disposable electrochemical strip can be used in this embodiment. A diagrammatic representation of the disposable electrochemical strip is shown in FIG. 13.

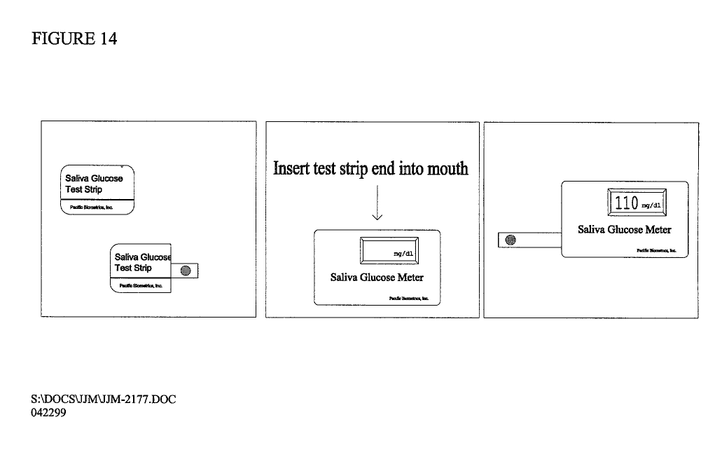

For the monitor-read disposable electrochemical glucose assay, a simple wettable “skinned” membrane is used over an osmotic driver impregnated wicking layer that interfaces intimately with the electrochemistry. A series of enzyme and electrochemical layers (applied as coatings, filters or membranes) is configured to lie under the osmotic driver layer but on top of the dielectric layer covering the microelectrodes printed on the collection/assay device. As sample passes the membrane layer, following citric acid stimulation, it will penetrate the layers of the device to produce electrons. The electrons will be detected by a glucose monitor and converted to a visual display. A range of sample from 10 to greater than 30 μl of sample will be required for a valid result. Current blood glucose electrochemical strips of comparable design employ a similar volume. Additionally, owing to the direct contact of layers and the inherent wicking design of this paper-based disposable, it is expected that sample collection can take less than 30 seconds. FIG. 14 shows the usage and interpretation of readings obtained from an individual under going insulin therapy.

Interpretation of results, expressed in mg/dL blood equivalents (or directly as blood level in the case of a blood measurement in a combo salivalblood assay) can be provided by the monitor. The monitor will employ the appropriate algorithm(s) to allow conversion of saliva values to blood equivalents. The algorithm may also consider patient information such as age, sex, fasting or not, prior glucose levels, etc.

The following Examples demonstrates the present invention.

### EXAMPLE I

As observed by Borg and Birkhed (1988), saliva glucose in whole oral fluid rose and fell in concert with blood glucose from a finger stick following an ingestion of bollus glucose (25, 50, and 75 g) in non-diabetic volunteers. However, utilizing the present invention, duplication was achieved of the oral elevation by having subjects dissolve glucose tablets in the mouth, followed by expectoration without swallowing. In this Example, there was no, or at least only minor, elevations in blood glucose. It can be concluded that following absorption of glucose by oral mucosea, tissue becomes a dominant source or sink of salivary glucose. It can take two hours for saliva glucose to reach precontamination baseline values. Various rinsing protocols using water, concentrated sodium chloride, and glucose-free astringent mouthwashes only modestly reduced the time to baseline. It was also determined that routine meals not regulated for content have the same effect as glucose tablets, though the degree of oral contamination was reduced compared with tablets or concentrated liquid glucose.

### EXAMPLE II

It was previously hypothesized that saliva glucose could be detected even in periods of hypoglycemia given the development of a highly sensitive glucose assay. When such an assay was perfected, it was used to confirm the existence saliva glucose threshold, though at about one-half the blood glucose concentration claimed by Reuterving et al. (1987). The confirmation of the threshold of 70 to 100 mg/dL in eight non-diabetic subjects led to the investigation of saliva glucose levels in normal to hyperglycemic persons.

A study in 18 diabetic subjects was initiated, the subjects being screened by finger stick to ensure the existence of the study criteria of greater than 250 mg/dL. Subjects contributed whole saliva samples and samples collected by a device made in accordance with the present invention. Subjects also provided venipuncture blood for measurement of glucose by the reference method. This method uses the enzyme hexokinase to phosphorylate (using ATP) glucose to glucose-6-phosphate. Glucose-6-phosphate is next converted to 6-phosphogluconate with reduction of NADP′ to NADPH, the latter reaction read with a spectrophotometer (340 nm) after a specified period of time; the amount of NADPH produced is proportional to the amount of glucose in the deproteinized sample.

The data reveal a correspondence between finger prick blood glucose and glucose derived by the device made in accordance with the present invention when blood and saliva samples are taken at the same time. The correspondence with venipuncture glucose is also high, shown in FIG. 12.

### EXAMPLE III

A highly sensitive assay for saliva glucose was derived. Table I lists most visible wavelength chromogens, investigated, identifies the limits of glucose detection (+2 standard deviations of blank in phosphate buffer), and tabulates time to complete assay (high standard OD 1.2-1.8). These assays were done in solution (96 well plate, sample volume 100 μL) at 37° C. with samples added last.

TABLE 1
Visible Wavelength Chromogens in GO/HRP
Glucose Assays Investigated

| | | | Time to |
|---|---|---|---|
| | Sensitivity | Response | Completion |
| *Coupled Reagents: | mg/dL | OD/mg/dL | minutes at 37° |
| MBTH- | | | |
| DMAB | 0.04 | 0.18 | 15 |
| CTA | 0.06 | 0.13 | 20 |
| 3,6 CTA | 0.12 | 0.08 | >30 |
| 5,7 CTA | | 0.12 | 25 |
| 4-AA- | 0.16 | 0.14 | 15 |
| 4-HBS | | | |
| *Single Reagents: | | | |
| O-Di | 0.29 | 0.04 | >30 |
| OPD | 0.16 | 0.08 | not done |
| 5-AS | 0.25 | 0.02 | >30 |
| ABTS | 0.15 | 0.08 | 15 |
| TMB | 0.22 | 0.11 | 20 |

*Names of compounds used in Appendix I. Assays done in phosphate or Tris buffers.

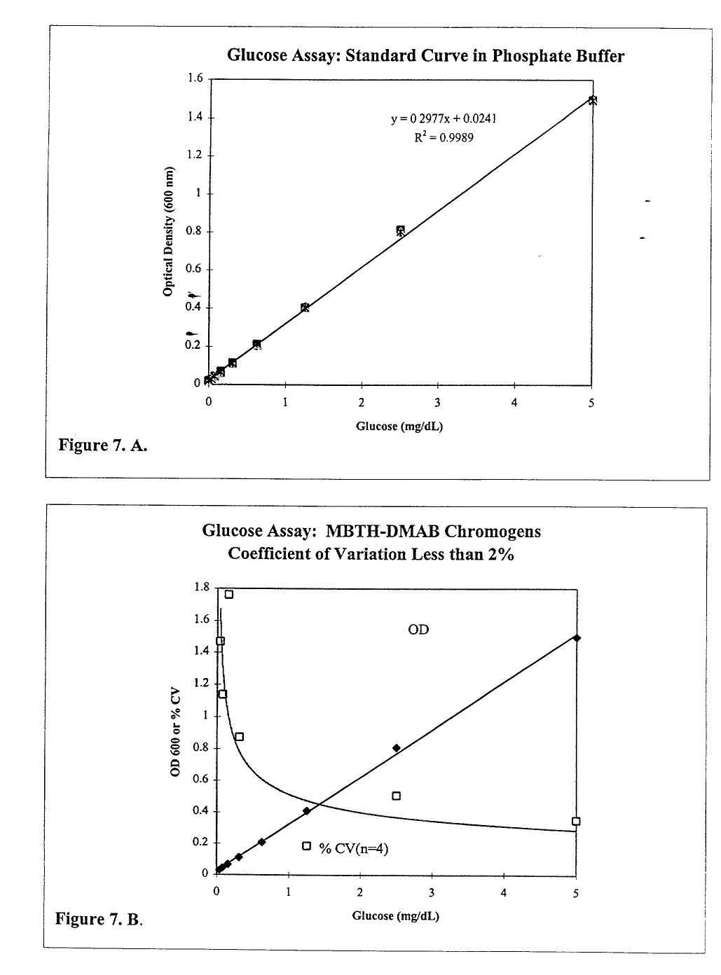

FIG. 6A summarizes a subset of the visible chromogens used with glucose oxidase-peroxidase in development of a more sensitive glucose assay. Glucose was spiked into two different matrices: 20 mM phosphate buffer (pH 7.0), and whole saliva processed as described above but without heating to 100′×10 min (saliva pH, 6.9). The whole saliva used was donated by a single fasting individual and did not have detectable glucose before spiking in any of the assays. The MBTH system, compared to other chromogens, showed the greatest sensitivity and steepness of response with acceptable linearity in the target dynamic range. FIG. 6B emphasizes the performance of various systems in saliva and shows that the MBTH (in this case, with CTA) system is superior to others (and also that it behaves in saliva as in buffer, with the exception that the limit of detection is slightly higher).

FIG. 7 shows the results in the final modification made to the MEBTH assay; this was in linking color generation to reduction of MEBTH and DMAB. This assay could detect 0.04 mg/dL glucose at the two standard deviations criterion (0.06 mg/dL in saliva). The percent coefficient of variation was less than 2% below 1 mg/dL and less than 0.6% when glucose exceeded 1 mg/dL (FIG. 7B). Table 2 summarizes composition and methods used for the GO/HRP-MBTH/DMAB glucose assay in the remaining studies shown.

TABLE 2
Composition of GO/MBTH Glucose Assay

| Solution | Enzyme | Chromogen | Buffer |
|---|---|---|---|
| 1 | Horseradish Peroxidase | DMAB* | 100 mM PO4 |
| | 12.5 U/mL | 30 mM | pH 7.5 |
| 2 | Glucose Oxidase | MBTH** | 100 mM PO4 |
| | 37.5 U/mL | 1.5 mM | |

100 μL Solution 1;20 μL Solution 2; Incubate 15 min at 37° or 25 min at room temperature. Read OD @ 600 nm.
*3-dimethylaminobenzoic acid
**3-methyl-2-benzo-thiazolinone hydrazone (dissolved in methanol at 15 mM)

### EXAMPLE IV

In this Example, a series of experiments were performed to learn if whole saliva could be processed in a manner that would reduce variability and improve accuracy in assay of glucose. As summarized above, it was determined that both goals could be achieved only after treatment of saliva using four separate procedures: sonication, mucoprotein precipitation using freeze-thawing, precipitation of soluble proteins using 10% TCA (trichloracetic acid), and heating processed saliva to 100° C. for 10 minutes. In most cases, each step requires its own subset of manipulations, such as centrifugation or readjustment of pH to assay optimum.

Table 3 shows one experiment in which one sample of whole (unstimulated) saliva was processed according to the sequence outlined. Separate aliquots were spiked with glucose at 1.5 mg/dL or 0.1 mg/dL before sample treatment, and processed in parallel. After each processing step, the product was assayed using the MBTH/DMAB glucose assay. The % CV for each assay (4 replicates A) is shown in parentheses to indicate variability.

TABLE 3
Effect of Processing Whole Saliva on Accuracy of Glucose Assay

| | 0 mg/ | | | | | |
|---|---|---|---|---|---|---|
| | dL | | 1.5 | | 0.1 | |
| Sample | Spike | (% CV) | mg/dL | (% CV) | mg/dL | (% CV) |
| Whole | 1.71 | (18.3) | 3.63 | (28.1) | 1.92 | (25.0) |
| Sonicated | 1.94 | (20.40 | 2.79 | (16.3) | 1.37 | (14.6) |
| Freeze-Thaw | 0.75 | (10.06) | 1.48 | (12.9) | 0.86 | (13.5) |
| TCA Ppt | 0.64 | (5.6) | 1.63 | (8.2) | 0.68 | (11.5) |
| Heat 100° | 0.62 | (5.8) | 1.77 | (7.3) | 0.70 | (9.9) |
| % Expected in | | | 83.5% | | 97.2% | |
| Final Step: | | | | | | |

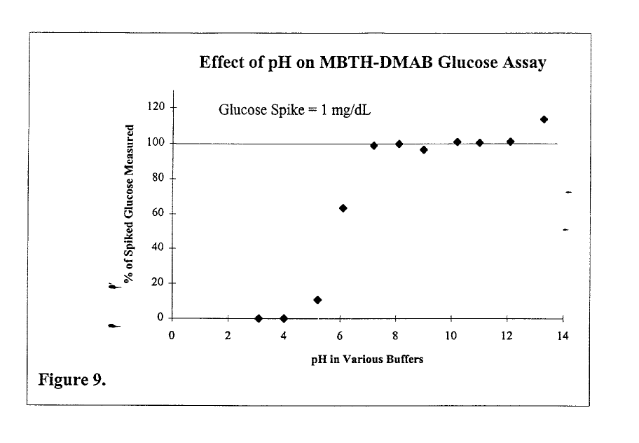

Spiked glucose was measured in saliva with approximately 80-110% recovery. However, the saliva of individuals is quite different with less viscous samples being less variable and requiring less processing. Capacity of the present invention to obtain a sample which accurately reflects, at equilibrium, whole saliva glucose was examined. One such experiment is shown in FIG. 8. In these four nondiabetic subjects, glucose collected and measured in accordance with the present invention reached approximately (±12%) the whole saliva concentration in 26 minutes (each subject placed two devices in the mouth and these were removed at 12 minutes and 26 minutes: The arrowheads on the right indicate the glucose concentration measured in whole saliva collected between minutes 26-29). These results showed that the contents collected did equal concentration in whole saliva, though the time required was somewhat longer than the earlier estimate. The longest times required seemed to be in those individuals with the thickest whole saliva.

Subsequently, it became possible to obtain a less viscous saliva in all subjects by stimulation with citric acid. With citric acid, time to reach equilibrium with whole saliva glucose appeared to be reduced to 12 minutes.

Table 4 illustrates an experiment of the type described above in which three nondiabetic subjects and one diabetic subjects had two devices, made in accordance with 10 the present invention placed in the mouth, but on this occasion, following citric acid. When compared to whole saliva (collected after removal of the last device and reapplication of citric acid), most subjects showed glucose values from fluid collected by the subject device approximately equal to whole saliva by 12 minutes, but at least one required longer.

TABLE 4
Time to Reach the Equilibrium Glucose Concentration in a Device
Made in Accordance with the Present Invention Following Citric Acid
Stimulation of Salivation

| | Time | SalivaSac Glucose | End Whole Saliva Glucose |
|---|---|---|---|
| Subject | (minutes) | (mg/dL) | (mg/dL) |
| 1 | 12 | 0.85 | 0.94 |
| | 20 | 0.88 | |
| 2 | 12 | 1.14 | 1.43 |
| | 20 | 1.56 | |
| 3 | 12 | 0.43 | 0.52 |
| | 20 | 0.57 | |
| 4* | 12 | 3.27 | 3.67 |
| | 20 | 3.35 | |

Values are means of replicate determinations with Standard Errors less than 7.3% (SalivaSac) or 11.8% (whole saliva) of the mean.
*Diabetic subject

Stimulation of salivation promotes collection of a filtered sample, collected in accordance with the present invention, which reflects whole saliva glucose in less time than in unstimulated saliva. This advantage apparently originates from reduced viscosity which will increase diffusability of glucose. The deficiency in the large molecular weights mucopolysaccharides and mucoid proteins in stimulated saliva may also prevent “coating” of the sac membrane which could also interfere with flux of analyte.

Subsequent investigation unexpectedly showed that glucose in stimulated saliva (whole processed saliva or when collected by the present invention) also showed closer parallelism with blood glucose than did unstimulated saliva. Some explanation for this improved correspondence was gained by examination of certain biochemical properties of saliva which relate to mechanisms of secretion. In particular, it was investigated as to whether glucose absorption from the primary filtrate by salivary ducts might be minimized when flow through the ducts was maximized by stimulation. It was inferred that this is the case from the data presented in Table 5. It compares mean content of glucose, Na+, K+, soluble protein and total protein (and polysaccharides) in five individuals who contributed whole unstimulated and stimulated saliva within a 20 minute period. Soluble protein was measured using the Pyrogallol assay; the insoluble material was measured as dry weight of the freeze-thaw pellet.

TABLE 5
Concentration of Protein, Sodium, Potassium and Glucose in
Citric Acid Stimulated and Unstimulated Whole Saliva

| | Insoluble | Soluble | | | |
|---|---|---|---|---|---|
| | Protein | Protein | Na+ | K+ | Glucose |
| | (mg/mL) | (mg/mL) | (mM) | (mM) | (mg/dL) |
| Unstimu- | 7.5 ± 1.2 | 0.4 ± 0.2 | 10.4 ± 2.9 | 5.7 ± 1.9 | 0.6 ± 0.4 |
| lated | | | | | |
| Stimulated | 3.3 ± 0.9* | 0.5 ± 0.1 | 37.2 ± 5.9* | 8.8 ± 2.5 | 1.3 ± 0.5* |

Values are means ± SEM; n = 5. p ≦ 0.05, t-test.

Increased glucose concentration in stimulated saliva is consistent with reduced net reabsorption by the ducts. Likewise, the elevation in Na+ results from reduced time of exposure to ducted Na+ pump (Na−K−ATPase; 9). Stimulation of flow rate through the ducts would reduce net effect of any reabsorptive systems. The reality of a glucose reabsorptive system is also supported by existence of the saliva glucose threshold; the reduced amount of glucose diffusing from plasma when its concentration is low can apparently be completely cleared by the duct, provided flow rate is sufficiently slow.

Interestingly, the concentration of soluble protein is not significantly effected by stimulation, whereas insoluble materials are reduced. The reduced components are in the viscous, sticky material normally precipitated (in our method) by freeze-thawing and centrifugation. Its lower content can be observed in the “watery” saliva elaborated immediately upon stimulation. Soluble protein (to the extent it can be discussed as single class) is not lowered by stimulation; apparently secretion of some macromolecules is matched to the volume discharged, and others (especially the larger moieties) are not.

There is no ready explanation for the elevation in K+ upon stimulation. (3). It seems reasonable that with a reduction in reabsorption, saliva glucose will more precisely reflect the concentration of glucose deposited in the primary filtrate of salivary secretions. And this concentration will, in turn, be set by the free glucose concentration in plasma from which saliva glucose is ultimately derived.

The performance of the GO-HRO-MBTH/DMAB assay in the device of the present invention matrix following the dissolution of the NaCitrate osmotic drives was next examined. The focus was in the pH of this medium and the possible consequences of the elevated sodium ion and citrate concentrations. As sodium citrate is a weak base, it was found that in most subjects, pH in the device varied between 6.9 and 8.2. This is an important finding because the pH of the stimulated whole saliva is typically between 2 and 4, an effect of the acidic stimulant. Thus, Na₃Citrate in the sac and citric acid in the stimulant are acting as the conjugate pairs of a buffer, the effect in the sac producing a pH in the optimal range of the enzyme assay.

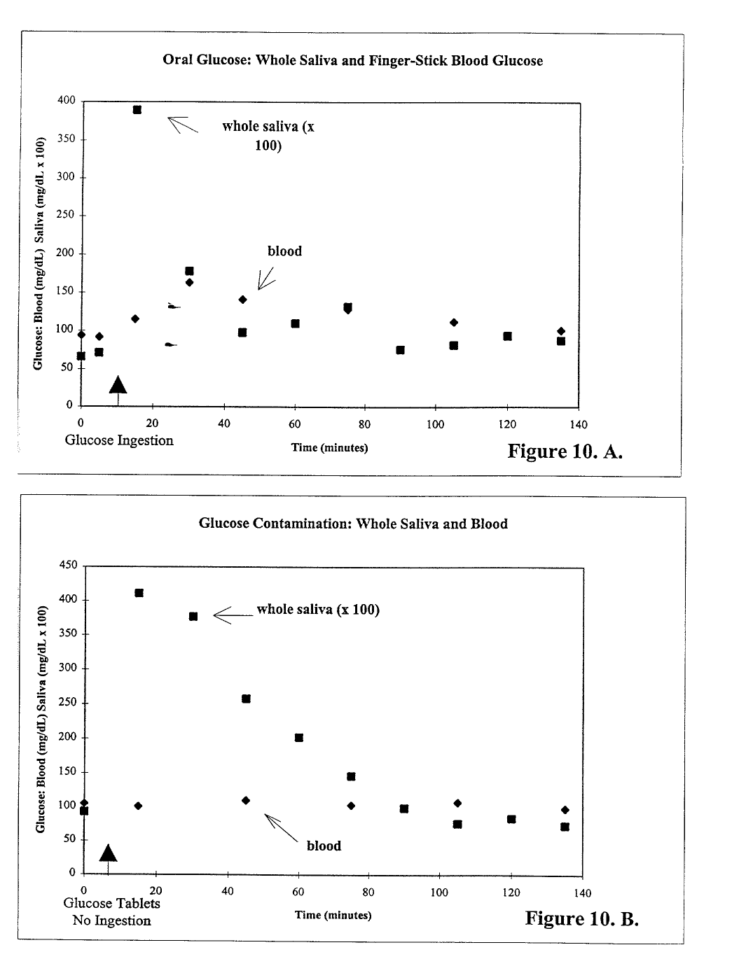

FIG. 9 shows the effect of pH on the Vmax of the assay when it is performed in 500 mM NaXitrate. The concentration of osmotic driver was arrived at by measuring Na+concentration (flame photometry) in several samples after the equilibration period of 20 minutes in the mouth. Na+ concentration was approximately 1.5 M(range, 1.25-1.8 mM) and the citrate concentrations was computed assuming that the ratio of Na/Citrate was maintained at 3. Conditions prevailing in the sample collected by the present invention are compatible with sensitive and accurate performance of the solution version of the strip assay.

Earlier research centered on development of a new sensitive glucose assay and in defining conditions in whole saliva and in samples obtained by the present invention that permitted accurate quantitation of saliva glucose. In the remaining results shown, the assay was used in human subjects to establish the basic feasibility of a saliva test as a potential substitute for blood tests.

### EXAMPLE V

Factors Influencing Diagnostic Specificity of Saliva:

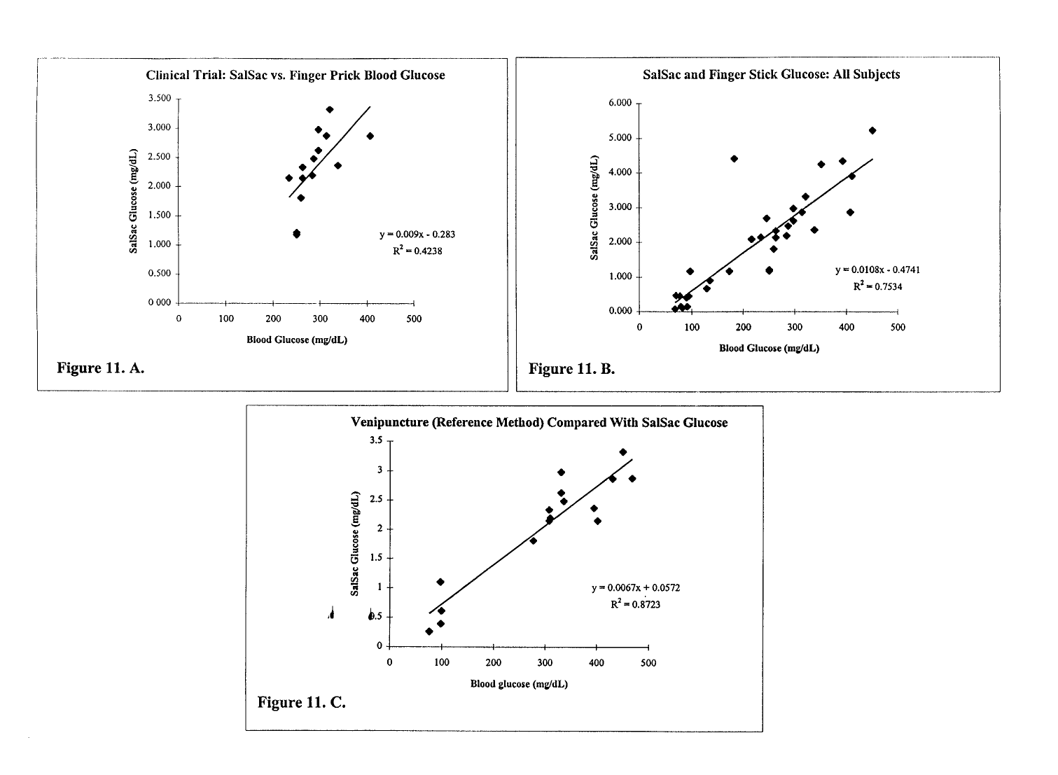

Two requirements of this noninvasive approach are that saliva glucose reflect blood glucose, and that the time lag in saliva be limited to minutes. The latter point is proven in the literature (Reuterving et al., 1987). The findings of Borg and Birkhed (1988) in which they showed a rise in saliva glucose following oral ingestion of glucose can be explained as an artifact of mucosal contamination which ostensibly duplicates the elevation in blood. The basis of the skepticism was that previous papers reported levels of saliva glucose that exceeded values observed and reported herein in subjects, even when blood glucose was relatively high. These are shown in FIG. 10A which illustrates the rise in saliva and blood glucose in one nondiabetic subject undergoing a modified Oral Glucose Tolerance Test, in which 50 g of glucose (in 200 mL H20))was taken orally and whole saliva and blood (finger-stick) collected for assay of glucose at 15 minute intervals. This experiment did not use the present invention as shown in FIGS. 1 and 2 as it was necessary to sample frequently at intervals less than the device equilibration period. Both blood and saliva glucose rise in the early period. FIG. 10B shows a similar experiment done in the same individual. In this case, however, the subject rotated two 5 g glucose tablets within his mouth for four minutes, and next expectorated undissolved tablets and saliva, and rinsed mouth once with water prior to contributing whole saliva and blood samples. In this case, there was a transient elevation in saliva glucose as in the earlier experiment, but this one was not paralleled by blood glucose.

It is evident that glucose contamination of tissues of the mouth, especially when oral glucose load is high, can be the dominant source of glucose measured in saliva. The same contamination could apply when glucose loading is reduced to the content in an average meal. Table 6 shows that glucose collected in accordance with the present invention (referred to as “SalivaSac”) tends to be higher in some individuals one to two hours after than immediately before lunch, even when blood concentrations increase only modestly between sampling periods.

TABLE 6
SalivaSac Glucose and Blood Glucose Before and After Lunch

| | Before | Before | | | |
|---|---|---|---|---|---|
| Subject | Saliva | Blood | Mg/dL | After Saliva | After Blood |
| 1 | <0.12 | 77 | | 1.22 | 103 |
| 2 | 0.18 | 89 | | 5.13 | 98 |
| 3 | <0.12 | 102 | | 1.38 | 107 |
| 4 | 0.43 | 101 | | 1.49 | 103 |
| 5 | 0.17 | 98 | | 0.51 | 132 |
| 6 | 0.49 | 96 | | 1.64 | 142 |
| 7 | 1.32 | 100 | | 1.26 | 74 |
| 8 | <0.12 | 82 | | 0.46 | 92 |

SalivaSac ® in mouth for 20 minutes after citric acid stimulation; blood glucose measured using the same finger-stick strips and monitor (One-Touch ®). Meals unregulated for glucose content; after lunch samples taken 1-2 hour after meal. 0.12 mg/dL was the LOD (2 standard deviations criterion) in SalSac samples at the time this assay was done.

### EXAMPLE VI

As discussed above, the threshold for saliva glucose is a blood glucose of approximately 100 mg/dL or less. A study in hyperglycemic individuals in which whole saliva and glucose collected by the present invention were compared with blood glucose in finger-stick and venipuncture samples. These data are presented as evidence in support of the contention that the present invention is feasible when blood concentrations are normal to elevated. The entrance criteria for this study was a blood glucose of greater than or equal to 250 mg/dL. Subjects were not required to fast overnight, but were asked to refrain from eating for the three house before samples were taken in mid-morning or mid-afternoon. Adult subjects meeting criteria placed a device made in accordance with the present invention in the mouth after stimulation with citric acid. The collection period was 20 minutes, after which subjects also donated whole saliva and venipuncture blood.

FIG. 11A shows glucose collected by the present invention plotted against finger stick glucose. Each subject used their own monitor to obtain the finger stick glucose value. SalivaSac and SalSac in the figures indicates use of the present invention. Variation in blood measurements by use of several monitors of unknown precision or calibration might have contributed to scatter in the correlation (Li et al., 1994). Nonetheless, there is a general correspondence between glucose and blood glucose. It is also evident that glucose collected by the present invention values in hyperglycemic subjects exceeded the typical concentrations observed in normoglycemic persons.

FIG. 11 combines the data obtained in the study of diabetics with data previously obtained using four nondiabetic and one diabetic subject. Each of the earlier subjects were sampled twice, once before lunch and once after, and both measurements are included in the figure.

FIG. 12 presents data from the same experiment with diabetic and non-diabetic subjects. In this figure, the correlation between saliva glucose collected by the present invention and glucose in venipuncture blood measured by the reference method (Hexokinase; see above) is shown. When blood glucose exceeds approximately 70 mg/dL (in the normal range), there is a close parallel between blood and filtered saliva. Data on FIGS. 11 and 12 show that whether blood glucose is measured by the finger stick method (as is typical among diabetics) or by venipuncture (as occurs in medical practice), the present invention obtains a saliva sample that corresponds with the blood values. As noted above, the precise nature of the computation to estimate blood glucose has not yet been determined, though its general form is shown by the equations in FIGS. 11 and 12.

In view of the above, it can be concluded that glucose in saliva is quantitatively related to glucose concentration in plasma from which it is derived. The relationship is only effective to individuals and situations in which blood glucose is greater than 70-100 mg/dL.

Further, the above data demonstrate the feasibility and utility of the subject method wherein, generally, blood glucose is monitored by most generally, stimulating salivary glands secretion of saliva into oral fluid, collecting a sample of the oral fluid, detecting an amount of glucose in the sample and then quantitating blood glucose level based on the amount of glucose detected.

Throughout this application, various publication are referenced by authors and years. Full citations for the publication are listed below. The disclosure of these publications in their entireties are hereby incorporated by reference into this application in order to more fully describe the state of the art to which this invention pertains.

The invention has been described in an illustrative manner, and it is to be understood the terminology used is intended to be in the nature of the description rather than of limitation.

Obviously, many modifications and variations of the present invention are possible in light of the above teachings. Therefore, it is to be understood that within the scope of the appended claims, reference numerals are numerals are merely for convenience and are not to be in any way limiting, the invention may be practiced otherwise than as specifically described.

### EXAMPLE VII

The apparatus and methods of the present invention were used to measure a subject's glucose levels by measuring glucose in the alternate body fluid saliva. The objective of this study was to demonstrate the clinical utility of, and benefits of, collecting a saliva ultrafiltrate for use in measurement of glucose content in an alternative matrix and to diagnose diabetes in tested individuals.

A total of 65 subjects were recruited and completed the study: 34 normal subjects and 31 subjects with clinically confirmed diabetes. Subjects fasted for 12 hours prior to testing and used a water only mouth rinse prior to testing. Salivary stimulation with citric acid was used immediately prior to saliva collection. Optimal processing and recovery of glucose in saliva was obtained using an embodiment of the apparatus of the present invention.

The embodiment used in the present Example was a device composed of a 2 cm diameter double membrane with a molecular weight (MW) cutoff of 60 kD held together by two plastic rings which snapped together. Contained within the membranes was 10 mg of sodium citrate, which acted as an osmotic driver. The device was placed in the mouth for 5 minutes collection time. The total volume of ultrafiltrate collected was 60 microliters.

Blood samples were collected using both venipuncture and finger stick methods. Blood samples were heparized. The finger stick sample was used for comparison to saliva in the results presented here. Glucose analysis of blood and saliva ultrafiltrate was made using the Yellow Springs Instrument (YSI), (Yellow Springs, Ohio), model 2700D chemical analyzer. In addition to blood and salivary glucose, pH was measured using an Orion (Beverly, Mass.) micro pH electrode. Potassium and sodium were measured with an Instrument Laboratories (Anaheim, Calif.) model 943 flame photometer.

The results from this study demonstrated the importance of salivary stimulation in the collection of a fresh sample. Differences in pH, potassium and sodium showed no significant effect on the relationship between blood and saliva. Demographic factors had no significant effects on the saliva/blood relationship.

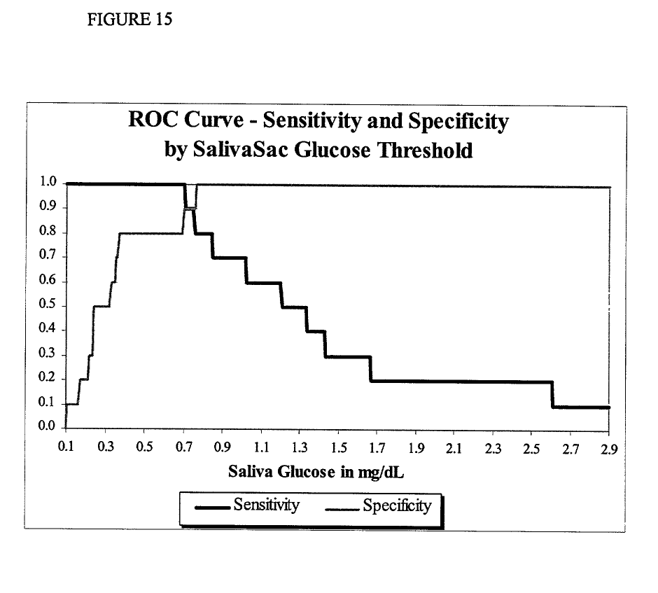

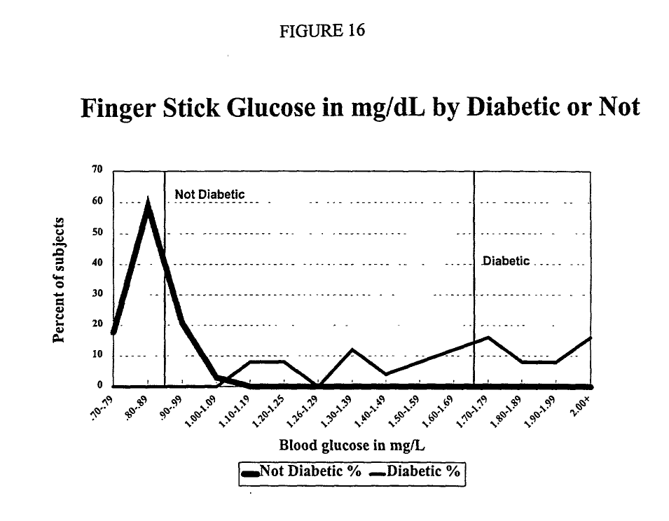

The results show that within each group, saliva is able to distinguish between subjects with diabetes and normal subjects. Saliva did demonstrate some overlap between the two groups. FIG. 15 demonstrates the results of the finger stick blood glucose values for patients with diabetes and normal subjects. FIG. 16 demonstrates the saliva glucose values for these same two distinct populations. The vertical bars indicate the mean values for each set. Note that the mean values for the saliva comparison are distinct, but with some overlap of results in the two populations.

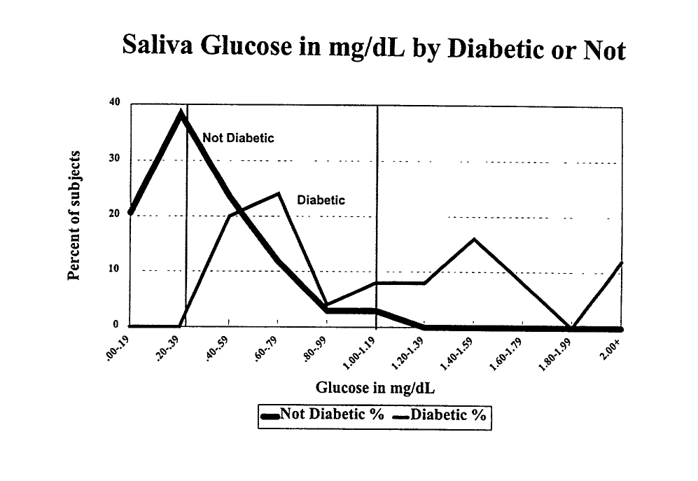

FIG. 17 shows the Receiver-Operating Characteristic (ROC) curve for clinical sensitivity and clinical specificity for saliva glucose. The data for Sac collection showed a 90% sensitivity at 90% specificity. Such analysis allows for the establishment of the cutoff values to be selected.

The results described above confirm the utility of the saliva system and the Sac as a method to improve the usefulness of saliva as an alternative matrix for diagnosing diabetes. The results also show the ability of the Sac to discriminate between normal subjects and subjects with diabetes as defined by their blood levels. Increased levels of glucose in saliva are fairly highly correlated to increased levels of glucose in blood.

These results also established that threshold levels can be set for subjects undergoing a saliva glucose-screening test for diabetes. These threshold levels can be used to classify subjects as (1) not at risk, (2) potentially at risk (between 110 mg/dl and 120 mg/dl in blood) and (3) at risk. The threshold levels can be adjusted according to subject age groups or other factors.

### EXAMPLE VIII

This Example discusses the use of the devices of the present invention to monitor glucose levels and thereby diagnose and monitor glucose-related disease states, such as diabetes. The results below relate to a visual colorimetric assay with finite fixed yes/no cutoffs at particular glucose levels with indications of normal, impaired or diabetic status for both normal and diabetic patients in addition to criteria for the electrochemical assays. The latter requires a closer correlation to establish relative saliva-to-whole blood equivalents in addition to establishing the biological correlation between the two fluids through either a patient-tracking or population-based algorithm.

In the preliminary trial, data were collected from 14 “subjects” and 19 “others”. Although there is incomplete documentation of this trial, the 14 subjects were diabetics recruited to the study, whereas the other 19 were non-diabetics as obtained from laboratory volunteers, neither group had fasted prior to testing. Twenty mg of citric acid crystalline powder was used to stimulate salivation. Several different calculations were done from the saliva samples derived from the 14 subjects, including pH adjustment. A solution based calorimetric assay was developed for this study. The blood glucose was measured as (1) mg/dL fmgerprick; and (2) venipuncture, both using the referenced method Spectrum HexoKinase glucose assay. Although the two measures of blood glucose were almost totally uncorrelated, the mg/dL fingerprick measure was used since it was most comparable to the blood glucose measures used in the later study.

Regression Analysis

Bivariate scalar (Pearson) correlation coefficients showed that the best fit between regression this translated to an unadjusted R² of 0.424, and an adjusted R² of 0.376 (the adjustment accounts for the small sample size) and the result was statistically significant at the 0.01 level. A better result was obtained using the bivariate ordinal (Spearman) correlation coefficient. For this statistic, the correlation was 0.829 (significant at the 0.001 level). The R² of 0.687 indicates that 69 percent of the rank-ordering of blood glucose is explained by the rank-ordering of saliva glucose.

The addition of the other 19 patients increased the size of the correlation coefficient, largely because it provided values at the lower glucose levels. For these subjects the only data available were the Orig splc saliva glucose measure and the mg/dL fingerprick blood glucose measure. By combining data for these 19 people with the 14 “subjects” data were generated for 33 people. Among these 33 subjects, the scalar bivariate correlation was 0.868, producing an unadjusted R² of 0.753 and an adjusted R² of 0.745. The ordinal correlation was nearly identical, with an R of 0.882 and an R² of 0.778. These results are summarized in Table 7 on the following page.

TABLE 7
Correlation Between Glucose Measured in Saliva and Glucose Measured in Blood Over Several Clinical Trials

| | | | | | | | | Saliva | Membrane | SalivaSac |
|---|---|---|---|---|---|---|---|---|---|---|
| | | | | | Study | Data | SalivaSac | Collection | MW cutoff | Sample |
| Study | N | Fasting | Diabetic | Stimulation | Setting | Adjustment | Used | Time | (daltons) | Pooled |
| A₁ | 14 | − | + | 20 mg | Local | pH | Old | 20 min. | 10,000 | No |
| | | | | powder | Clinic | | | | | |
| A₂ + A₁ | 19 | − | − | 20 mg powder | Lab | pH | Old | 20 min. | 10,000 | No |
| | | | | | Volunteer | | | | | |
| B | 50 | + | 30 diabetic | 10 mg liquid | Seattle | None | New | 2 min. | 60,000 | Yes |
| | | | 20 normal | | (controlled) | | | | | |
| | | | | | | yes* | | | | |
| Combined | 78 | | | | | None | | | | |
| Combined | 78 | | | | | yes* | | | | |

| | Pearson Scalar | | | | |
|---|---|---|---|---|---|
| | Correlation | | | Spearman Ordinal | |
| | | | Adj. | Correlation | |
| Study | R | R¹ | R² | R | R² |
| A₁ | .691 | .421 | .376 | .829 | .687 |
| A₂ + A₁ | .868 | .753 | .745 | .882 | .778 |
| B | .542 | .294 | .277 | .610 | .372 |
| | .737 | .543 | .498 | na | na |
| Combined | .810 | .656 | .652 | .805 | .648 |
| Combined | .862 | .743 | .736 | na | na |

*for known covariates

The second more extensive clinical trial collected data from 50 subjects, 30 of whom were self-reported diabetics, and 20 of whom were not. This trial differed from the earlier one in that (1) there was suboptimal gland stimulation prior to the collection of saliva, (10 mg equivalent of citric acid solution sprayed at the back of the throat); (2) saliva was collected for a shorter duration of time (2 minute miniaturized Sac); (3) the saliva results as reported were not corrected for pH; and (4) Sac samples were pooled to obtain sufficient volume for analysis.

The clinical trial design involved the collection of a blood fingerstick sample upon entry followed by three, three-minute saliva sacs and then a venous blood draw; this in turn was followed by the collection of whole saliva (for processing by ultra-filtration in the lab), followed by a final fingerstick. The initial and final fingersticks were subjected to One-TouchTm (LifeScan Incorporated; Milpitas, Calif.) and Yellow Springs International (YSI) measurements at the time of collection. In addition, the patient's last One-Touch™ results were also obtained. The venous blood was processed for testing by the Spectrum HexoKinase assay. Sac samples were collected, pooled and stored frozen until tested. All Sac samples were processed on the same day using the YSI, Actimed (Burlingham, N.J.) C3 assay and liquid MBTH colorimetric assay used in the earlier study and discussed above. Appropriate demographic data was collected.

Initially all of the blood glucose measures were correlated to see what they looked like. All of the blood measures are highly intercorrelated, although we do not have data for all persons for all blood measures, so the number of variables available was more limited that it appeared at first.

The “finger prick YSI mg/dL” readings were treated as the “gold standard” and regressed against each of the other blood measures to search for outliers. There were three cases which exhibited significant change in the initial and final glucose reading, and these cases were consistent outliers: They are 59, 63 and 69. Ultimately patients 59 and 69 were deleted from the analysis for reasons detailed below.

All the measures of saliva glucose were then correlated. There is considerably less correlation among these variable than there is among the blood measurements. In particular, the “raw” scores (represented as raw instrument readings) are, as expected, uncorrelated with all other measures. The measures are highly correlated amongst themselves, but not with the other measures. The one variable that stands out as having the greatest number of high (>0.50) correlation coefficients with all other saliva measures is “YSI calc. gluc.” which was used as the gold standard for saliva. Since the “YSI calc. gluc.” was clearly the most highly correlated measure of saliva glucose, values were generated for the five missing cases by means of a regression equation using data for all other patients for whom complete data were available

Individuals whose blood and/or saliva glucose values seemed suspect were then searched for. Ultimately, five patients were dropped from the analysis, as follows:

59 is one of those who had taken glucose 1.5 hours before the test and he had very different initial and fmal glucose readings, so that seems to be evidence that he should be eliminated from the database.

62 is the only Pacific Islander in the study and she was the only patient who did not provide information on how long she had fasted, nor did she provide information when her last insulin was taken. She too was eliminated from the database.

66 had by far the lowest measured glucose in saliva despite a rather high blood glucose reading, but she was one of the people who had a potentially contaminated sample, and she was also a person who did not produce enough saliva for all tests.

69 had take insulin three hours before the test and had significantly different initial and final glucose readings.

78 had taken insulin within three hours and had a potentially contaminated sample of saliva, and she too was therefore eliminated from the database.

Regression Analysis

Prior to removing the above five cases, each saliva measure was regressed against each blood measure to search for the highest unadjusted correlation. Without any adjustments the best bivariate correlation between any blood and any saliva measure was the 0.505 between “finger prick YSI mg/dl” (the gold standard for blood) and the “YSI cale. gluc.” which had been used as the gold standard for saliva. This produced an adjusted R² of 0.255 and an adjusted R² of 0.239. The ordinal Spearman correlation coefficient was 0.566 (R² of 0.320).

After deleting the above five cases, the regression of the saliva glucose measure “YSI cale. gluc.” was run against the blood glucose measure “finger prick YSI mg/dl.” The Pearson correlation coefficient increased slightly to 0.542, producing an unadjusted R² of 0.294 and an adjusted R² of 0.277. The Spearman ordinal correlation coefficient was 0.610 (R² of 0.372).

Saliva glucose was then regressed against blood glucose, controlling individually for all demographic and biological variables. Several of the variables, including potassium, glyccrol, Na, volume, and volume/time were rechecked. As the criterion for keeping a variable as a covariate, the size of the R² -change induced by the addition of that variable was required to be statistically significant at the 0.05 level. Ultimately, there turned out to be three statistically significant covariates:

1) whether or not a person was diabetic (the relationship is stronger for non-diabetics);

2) whether or not a person had taken non-water liquids before the test-those who had consumed some other type of liquid prior to the test had a better relationship than those who had not, supporting the theory that the failure to stimulate the glands in this second round of testing may have contributed to the poorer result. The liquid variable was also measured in two other ways—water as the only liquid, and any liquid taken including water. However, neither of those two aspects of liquid-taking affected the overall relationship. The important factor appeared to be the intake of some liquid other than water. The third important factor was the pH level-this was an interesting finding since the saliva data in the original small sample were corrected for pH, but the data in this second study was not. It is also of interest to note that pH was only a statistically significant factor in the presence of the control for diabetes. Since the measure of pH in this study was somewhat approximate, this is a factor to measure more carefully in follow-up studies. All other variables were tested to see if they too would be significant in the presence of the diabetics variable, but pH was the only variable for which that was true.

Taking those four covariates into account, and without the five patients listed above, but with the imputed data for the patients who were missing YSC cal gluc results, the adjusted R² was found to be 0.498. This is a substantial rise from the 0.277 that was obtained without the covariates being taken into account. These results are shown in Table 7.

Combining The Two Datasets

In this section, a mini meta-analysis was conducted combining the data from the earlier and more recent studies, on the assumption that the finger prick YSI blood glucose data and the saliva YSI glucose data are comparable for the two series of data.

The two data sets have data for a total of 78 subjects. The Pearson correlation between blood and saliva glucose is 0.810, with an unadjusted R² of 0.656 and an adjusted R² of 0.652 (also see Table 7).

Regression Analysis

Regressing saliva glucose against blood glucose produced an adjusted R² of 0.652. Since the bivariate correlations indicated that the coefficient between blood glucose and being a diabetic was significant, but less than 0.400, this was included as a covariate. The result is a somewhat higher adjusted R² of 0.736.

ROC Analysis

ROC analysis measures the performance of a marker (in this case glucose as measured in saliva) in the identification of another condition (in this case glucose as measured in the blood). It evaluates the combination of the true positive rate (sensitivity-the probability of correctly detecting the condition of interest among subjects with the condition) and false positive rate (1 minus specificity, which is the probability of correctly ruling out the condition among subjects without the condition). In general, the greater the cumulative area under the ROC curve, the better is the combination of sensitivity and specificity and the more reliable is the threshold value of the marker at correctly classifying a subject according to the underlying condition. The optimal threshold level is that which simultaneously maximizes both sensitivity and specificity. With relatively small sample sizes, there is a confidence band around the optimal threshold and the width of that band will increase with a decrease in sample size, all other things being equal.

The American Diabetes Association suggests that blood glucose greater than 126 is the threshold for being diabetic, whereas levels of 110 through 126 represent an impaired level, and less than 110 is unimpaired. Among the 33 people in the earlier study, those with low blood glucose all had levels below 110, whereas those with high glucose all had levels above 126; there were no people in the impaired category between 110 and 126. We therefore defined those people with blood glucose above 126 as “diseased” and those with 126 or less as “non-diseased.” These values assume that the subject was fasting, the blood glucose ranges are less than 140 (normal); 140 to 200 (impaired); and over 200 (diabetic). Information about fasting is limited in these data to the 45 people in the second study. All of them were asked to fast a minimum of 8 hours before being tested, and all but one reported at least 8 hours of fasting. The one who reported fewer than 8 hours fasting had a measured level of blood glucose of 80 mg/dl, so she would have been placed into the normal category whether or not she had fasted.

The Analysis

It is reasonable to begin the ROC analysis with the greatest amount of information possible−all 78 people in the combined studies. Analysis was conducted using a spreadsheet made available by Matthais Greiner at the Freie Universitaet in Berlin, Germany (htt://www1.vetmed.fu-berlin.de/in proj2.htm//Seroepimiology). The analyses are summarized in Table 8.

For all subjects in the two combined studies, the area under the curve (theta) is 0.821, indicating a reasonably high level of predictability in classifying a person's blood glucose as being under or over 126 mg/dl, based on glucose as measured by the SalivaSac. The optimal threshold value for glucose in saliva is based on the non-parametric measure since the data are not normally distributed. The point estimate is 0.584 (with a 95% confidence interval from 0.42 to 0.69). At this value, the sensitivity and the specificity of the test are both 0.80.

For this particular kind of screening test, sensitivity is probably more important than specificity. A person is not worse off in terms if health if the test incorrectly puts them in the high glucose category, but they may suffer if the test incorrectly puts them in the normal category and they do not seek treatment because of that false negative reading. Thus, if we look at the threshold that maximizes sensitivity and specificity but seeks the highest value of sensitivity, then we will have achieved the goal. For all subjects for both studies combined, that value lies between 0.489 and 0.531, a range in which sensitivity is 0.90 and specificity is 0.81. In the remaining discussion, values are reported in the summary Table 8.

TABLE 8
Results of ROC analysis using a Blood Glucose threshold value of
126 mg/dl

| | Optimal threshold | | | | |
|---|---|---|---|---|---|
| | for saliva glucose | | | | |
| | based on higher | | | | |
| | sensitivity than | | | | Theta |
| | specificity | | | | (area |
| | Lower | Upper | Sensi- | Specifi- | Under |
| Sample | value | value | tivity | city | curve) |
| All subjects, both | .489 | .531 | .90 | .81 | .82 |
| Studies combined; | | | | | |
| n-78 | | | | | |
| Diabetics, both studies | .434 | .515 | .87 | .63 | .74 |
| combined; n-39 | | | | | |
| Non-diabetics, both | .629 | .629 | 1.00 | .93 | .92 |
| studies combined; n-39 | | | | | |
| All subjects, first | .553 | .676 | 1.00 | .89 | .93 |
| study; n-33 | | | | | |
| Diabetic, first study; | | <1.179 | 1.00 | n/a | n/a |
| n-14 | | | | | |
| Non-diabetic, first | .471 | .676 | 1.00 | .89 | .89 |
| study; n-19 | | | | | |
| All subjects, second | .458 | .524 | .82 | .79 | .78 |
| study; n-45 | | | | | |
| Diabetic, second | .431 | .524 | .81 | .67 | .71 |
| study; n-25 | | | | | |
| Non-diabetic, second | .538 | .616 | 1.00 | .89 | .97 |
| study; n-20 | | | | | |
| Last liquid is none or | .449 | .523 | .70 | .71 | .70 |
| water, second study; | | | | | |
| n-34 | | | | | |
| Last liquid is other | .431 | .550 | 1.00 | 1.00 | 1.00 |
| than water, second | | | | | |
| study; n-11 | | | | | |
| SUMMARY (limits) | .431 | .676 | | | |

The only covariate available for both studies combined is whether or not a person is self-reported to be a diabetic. The optimal threshold for this application, falling between 0.434 and 0.515, in which range the sensitivity is 0.87 and the specificity is 0.63. For diabetes the area under the curve is 0.74, indicating somewhat less accuracy than for all subjects.

The non-diabetic subjects were more predictable, as indicated by the area under the curve of 0.92. The optimal saliva glucose threshold is at a value of 0.629, where sensitivity is 1.00 and the specificity is 0.93.

We turn now to the earlier study only, looking first at all 33 subjects in that study. Table 8 above shows that the area under the curve is 0.93, and the optimal threshold values are between 0.553 and 0.676, in which range the sensitivity is 1.00 and the specificity is 0.89. In that earlier study, there were 14 subjects who were diabetic. However, since all of them had blood glucose above 126, it was not possible to calculate the area under the curve.

Nonetheless, we can note that the lowest glucose in saliva value was 1.179 so that represents a value above that which would be the optimal threshold.

There were 19 subjects in the first study who were apparently non-diabetic. As can be seen in Table 8 above the area under the curve is 0.89 and the optimal saliva glucose values are between 0.471 and 0.676, where the sensitivity is 1.00 and the specificity is 0.89.

Turning now to the later, second study, there are data for a total of 45 subjects. The area under the curve was 0.78, and as can be seen in Table 8, the optimal threshold values were between 0.458 and 0.524, where sensitivity was 0.82 and specificity was 0.67.

There were 25 subjects in the second study who indicated that they were diabetic. The area under the curve was 0.71, and the optimal threshold value was between 0.431 and 0.524, where sensitivity was 0.81 and specificity was 0.67.

Twenty of the subjects in the second study were normal—non-diabetic. The area under the curve for this group was 0.97, and only one person in this group had blood glucose above 126 mg/dl. The optimal threshold values based on this group would be between 0.538 and 0.616, where the sensitivity is 1.00 and the specificity is 0.89.

The covariates of pH and liquid were taken. The value of pH did produce any different set of results from those shown above. However, the other covariate-last liquid taken-did produce intriguing results. For those who had taken no liquid or only water prior to producing the saliva sample, the results were not very accurate. The area under the curve was 0.70, sensitivity 0.7 and specificity was 0.71 for the optimal thresholds between 0.449 and 0.523. On the other hand, for those who had taken some liquid other than water, there is a perfect classification of those with high and low blood glucose based on the saliva results. The area under the curve is a perfect 1.00, and between the saliva glucose threshold values of 0.431 and 0.550 the sensitivity and specificity measures are both 1.00. To be sure, the number of subjects is small (n-11), but these results reinforce the potential importance of stimulating the glands prior to obtaining the saliva sample.

The final ROC analysis assumes that the earlier study may have been done without asking people to fast (we know that all but one of the subjects in the second study complied with the fasting regimen). For non-fasting, the ADA thresholds of blood glucose are less than 140 is normal, 140 to 200 is impaired, and over 200 is diabetic. The cross tabulation of these categories of blood glucose by saliva glucose, indicates a close fit between these categories and the saliva measurements. An ROC analysis for these data using a dichotomy of 200 or less, and over 200 was conducted. This produced an area under the curve of 0.91, and a unique threshold value of 1.19 (glucose in saliva) with a sensitivity of 0.91 and a specificity of 0.92.

The results above show that the saliva test for glucose is related in an important way to the blood glucose level. The two factors do not track each other in a perfectly truly scalar fashion, at least not as measured in these studies. However, it is clear that there is a high ordinal correlation-an increase in the measured glucose in saliva is fairly highly correlated with an increase in the measured glucose in blood. This ordinal relationship is especially captured by the use of the ADA cutoff of 126 mg/dL glucose in blood in which the ROC analysis showed that there is a reasonably high level of both specificity and sensitivity around the glucose in saliva threshold level of 0.55.

### EXAMPLE IX

The Examples above show that there is a high ordinal correlation between blood and saliva glucose levels-an increase in the measured glucose in saliva is fairly highly correlated with an increase in the measured glucose in blood. This ordinal relationship is especially captured by the use of the ADA cutoff of 126 mg/dL glucose in blood in which ROC analysis above showed that there is a reasonably high level of both specificity and sensitivity around the glucose in saliva threshold level of .55. The data from prior studies suggested that a follow-up study should be carefully monitored to include the following:

1. A larger number of subjects and an attempt should be made to seek out people who clearly are diabetic, are clearly not diabetic, and those who fall in the “impaired” middle category;

2. Respondents should be screened for fasting or assigned randomly to fasting and non-fasting groups to test the differences in the sensitivity of the saliva samples;

3. Respondents need to be screened for glucose intake prior to taking of blood and saliva;

4. It is especially important that subjects have proper saliva gland stimulation prior to producing their saliva samples; and

5. Researchers should also make careful pH readings of all subjects.

6. SalivaSac® samples should not be pooled. The recommendations from the prior studies were used as the basis for the third study. This third study was very carefully set up and controlled. The goal was not only to assess covariates but also to establish a difference in the salivary glucose values between diabetes and normals without experimental error. Some additional restrictions were placed on participant inclusionary criteria which a review of the literature indicated might be important.

The third clinical study encompassed the following considerations: a statistically significant “N” for the disease and normal group was used, based on a power analysis and all subjects fasted twelve hours. Patients were excluded if they had: Oral prosthetic devices beyond two bridges; Oral candida carriage; History of bacterial infection; History of gingivitis or periodontitis; and All patients were in the age group for a screening test (35-70 years).

The methodology used in the present Example was altered compared to that of the previous experiments. For example, stimulation methods were re-optimized and verified before the study and all study participants were properly stimulated. The study involved the sequential collection of two small and one large Sac in which the time of collection relative to stimulation was recorded. The time between stimulation and first sac collection was kept to a minimum (<10 seconds). Participants were not allowed to take beverages on the morning of testing and were prohibited from teeth brushing. Participants were allowed one tap water rinse no earlier than one hour prior to stimulation.

Further modification to the methodology included an adjustment of the the small saliva sac collection time to five minutes (versus three). Venous blood was drawn during large sac collection and whole blood fingersticks were conducted prior to stimulation and after large sac removal. The YSI 2700 was used for all glucose measurements. A wash step was found to be necessary between saliva samples. The use of calibrations on the YSI instrument prior to whole blood or saliva use was extensive. Whole blood fingerstick samples were run at the clinic immediately after collection. Plasma hexokinase glucose values were determined on venous draws. Plasma potassium values were determined. Concerning the saliva samples, salivary pH was accurately measured with a microelectrode. Saliva samples were not pooled. Saliva samples were tested for glucose, pH, K+ and Na+. The quality and accuracy of saliva measurements on the YSI instrument was carefully controlled relative to instrument calibration, background amperage and assay reliability and precision all with a single technician.

A total of 71 subjects were recruited for this follow-up study: 36 normal subjects and 35 diabetic subjects. All of these subjects met the inclusionary and exclusionary criteria and the protocol was administered as described in the previous section. Prior to analysis there were six subjects whose data were excluded from the study. Four of these subjects had saliva volume that was insufficient for measurement purposes (subjects 01-156, 01-158, 02-67, and 02-90). Three of these subjects were diabetic, and one was normal. Three of them were female and one was male; two were under 45 and two were older than 45. An additional two subjects were deleted because of problems created by the rinsing of the saliva sacs during the course of data collection. This was a problem unique to the study design and would not occur in the ordinary application of the saliva sac. The two subjects deleted were 01-38 (a normal Hispanic female) and 02-75 (a diabetic White non-Hispanic male). The subsequent analysis was performed using the remaining 34 non-diabetic and 31 diabetic subjects. The demographic composition of the two groups was as follows:

Gender: 71% of diabetics are female; 56% of non-diabetics are female.

Age: Average age of diabetics is 53; compared to 46 for non-diabetics;

youngest diabetic is 33, compared to 31 for youngest non-diabetic; oldest diabetic is 70, compared to 67 for non-diabetics.

Race: 84% of diabetics are white, non-Hispanic, compared to 94% of non-diabetics.

Height: Average height of diabetics is 66.6 inches, compared to 69.0 for non-diabetics.

Weight: Average weight of diabetics is 210 pounds, compared to 174 for non-diabetics.

Body Mass: Average pounds per inch for diabetics is 3.3, compared to 2.6 for non-diabetics.

The study measured glucose in blood in three different ways: (1) a fingerstick YSI glucose at time zero; (2) a fingerstick YSI glucose at time =45 minutes after start; and (3) a venous blood draw during the administration of the large Sac, with a subsequent YSI glucose assay. Each of these three was examined as a candidate for the gold standard measurement of glucose levels. All three measures were highly intercorrelated with one another (bivariate scalar correlations of 0.995 or higher), but only the second fingerstick YSI glucose (FS2gluc) discriminated perfectly between diabetics and non-diabetics. It also exhibited the highest correlation with each of the saliva glucose measurements, so FS2gluc was chosen as the gold standard, with one adjustment. The fmgerstick values consistently reported glucose values that averaged almost exactly 10 mg/dL less than the venous blood draw glucose values. The fingerstick glucose values for known diabetics were thus below the ADA thresholds, whereas the venous blood values were consistent with those thresholds. Accordingly, a constant of 10 mg/dL was added to the FS2gluc values for this analysis.

Four measures of glucose in saliva were produced by the study protocol, including a whole saliva sample, two samples from the small Sac (with a 5-minute collection interval) and one sample from the large Sac (with a 20-minute collection interval). Results for each of these measures were regressed against the gold standard of FS2gluc to assess the bivariate linear scalar correlation between each measure of glucose in saliva and glucose in blood. The results are reported in Table 9. The whole saliva sample was examined first to confirm its overall low correlation to blood glucose when not filtered through the Sac. As can be seen in Table 9, the glucose measured in whole saliva had a very low scalar correlation with the fingerstick blood glucose level—an adjusted R² of only 0.018, which is not statistically significantly different from zero. Each of the results using the Sac was significantly better than measuring glucose in whole saliva. The first small sac, measuring glucose two minutes after stimulation, produced the best overall results, as can be seen in Table 9. The R of 0.562 is associated with an adjusted R² of 0.304. As was true in the previous studies, the ordinal Spearman's measure of correlation produced a higher R² of 0.433. The Pearson scalar correlations suggest that the correlation between the saliva glucose and blood glucose declines somewhat as the time since stimulation increases. Thus, the best results were obtained with the first small sac, collected 2-7 minutes after stimulation; whereas the next best results were obtained with the second small sac, collected 7-12 minutes after stimulation; and the least best results were produced by the large sac, collected 12-32 minutes after stimulation. The remaining analysis uses the first small sac data as the most representative of the saliva glucose data.

The above results were all conducted without adjusting for covariates. The first set of covariates examined were the saliva-based measures of pH, K, and Na. None of these three elements made a statistically significant contribution to the relationship between saliva glucose and blood glucose. Demographic factors of age, gender, body mass, and whether or not the subject was diabetic were also examined as potential covariates. Sex was not a significant covariate, but both age and body mass were in the absence of the control for whether or not a person was diabetic. However, since older age and greater body mass are both highly correlated with being diabetic, the latter control washed out the importance of age and body mass. Thus, controlling for whether or not the subject was diabetic increased the R to 0.850 and the adjusted R² to 0.676, as can be seen in Table 9. These results are very similar to those obtained by combining the data for the two earlier studies (as shown in Table 9).

Since patient condition was the single important covariate in the relationship between Sac glucose results and fingerstick blood glucose results, diabetics were analyzed separately from non-diabetics. The results show that within each group, saliva is less able to distinguish the nuances of differences among subjects than it is able to distinguish between diabetics and non-diabetics. Thus, among non-diabetics, the scalar correlation coefficient between fingerstick blood glucose and SS1gluc was R=0.455, with an R² of 0.207. The Spearman's ordinal coefficient was actually slightly lower (R=0.378; R²=0.142). Among diabetics the correlation coefficients were lower. The scalar Pearson's R was only 0.091, which is not statistically significant from zero; and the Spearman's R was 0.193, which was also not significantly different from zero.

ROC Analysis

The ability of saliva glucose to distinguish clearly between diabetics and non-diabetics suggests that it has value for screening at threshold levels. This capability was tested using ROC analysis. These results are summarized in Table 11. The results show that at a threshold value of 0.580 (with a 95% confidence interval of 0.466 to 0.663, the sensitivity and specificity were both 0.76 and the area under the curve was 0.75. Much of the loss of accuracy was created by subjects near the 126 threshold. The threshold was lowered to 110 and these results are shown in Table 11. The results are slightly improved, but they are substantially the same as those for the 126 cutoff.

Given the potential indeterminacy of creating scalar glucose values from ordinal tests, the ROC was repeated after creating different sets of “gray zones” where the values are such that the user would be recommended to repeat the test to establish correct positioning below or above the threshold. The first such gray zone tested was the fingerstick blood glucose level of 110 through 126. The four subjects in that zone were set aside and the ROC analysis repeated. These results were nearly identical to the previous ones.

A second gray zone was then constructed using the ADA cutoff of 126 mg/dL±15% (which was the coefficient of variation around the blood glucose assay). This meant that eight subjects with a fingerstick blood glucose of 107 to 145 were in the gray zone and were dropped from the ROC analysis. As can be seen in Table 11, the results were slightly improved by the use of this gray zone, with the area under the curve rising above 0.80, and the sensitivity and specificity both being very close to 0.80.

Another way of viewing the results is to examine the levels of sensitivity and specificity that would be produced by using a three category range of scores for the interpretation of Sac values: (1) less than 0.440 =not at risk (comparable to blood glucose level less than approximately 110); (2) 0.440 through 0.663 =repeat test or use fingerstick blood test; (3) above 663 =at risk (comparable to blood glucose levels above approximately 126). Under that scenario, the results, as shown in Table 11, produce a sensitivity of 0.91, a specificity of 0.84, and an area under the curve of 0.85. These results would be even more impressive were it not for three discrepant cases (01-151; 02-86; and 02-85) of diabetics whose small sac readings were unusually low. It can be seen in Table 3 that without these cases the sensitivity rises to 1.00, while the specificity remains at 0.84, and the area under the curve remains at 0.93.

Discrepant Cases

Subject 01-151 is a 68 year old white male diabetic weighing 296 pounds (the heaviest of all male subjects). His medications include glucophage, lizinapro, imdur and provacol. His high blood glucose was confirmed by all three blood measures (fingersticks 1 and 2 and the venous draw) and his saliva glucose in the large sac would also have correctly placed him in the diabetic category, whereas both small sac readings were below the gray area as defined above. His sodium level as measured in the large sac was also higher than average, whereas it was not in the smallsac.

Subject 02-85 is a 66 year old white female diabetic weighing 181 pounds. Her medications include glucophage and sinthroid. Her high glucose level was confirmed in all three blood measures, but her reading on the first small sac was below the gray area. However, the reading on the second small sac would have placed her in the gray area, and the large sac reading would have placed her correctly in the diabetic category. Her sodium level was also above average.

Subject 02-86 is a 56 year old white female diabetic weighing 245 pounds. Her medications include vasotec and mevacor. Her high glucose level was confirmed in all three blood measures, but all three Sac measurements placed her in the not-at-risk category. There were no other apparent anomalies in her profile that would explain the discrepancy between the blood and the saliva results.

A comparison of the results from this Example with the results from Example XIII was made by examining the data in Tables 9, 10, and 11. It can be seen in Table 9 that the regression coefficient between saliva and blood glucose measure is very high (R² near or above 0.70) in Studies A+B Combined, as well as in Example IX when controlling for known covariates. The ROC results shown in Tables 10 and 11 show that the threshold range for distinguishing diabetic from non-diabetics using results from the Sac are consistently in the range from 0.440 to 0.663. Treating that threshold range as a gray area generates a lower bound below which there is a very high probability that subjects are correctly classified as non-diabetic (sensitivity ranging from 0.70 to 1.00 in the several studies), and an upper bound above which there is a very high probability that subjects are correctly classified as diabetic (specificity ranging from 0.63 to 1.00 in the several studies).

TABLE 9
Correlation Between Glucose Measured in Saliva and Glucose Measured in Blood Over Several Clinical Trials

| | | | | | | | | Saliva | Membrane | SalivaSac |
|---|---|---|---|---|---|---|---|---|---|---|
| | | | | | Study | Data | SalivaSac | Collection | MW cutoff | Sample |
| Study | N | Fasting | Diabetic | Stimulation | Setting | Adjustment | Used | Time | (daltons) | Pooled |
| A₁ | 14 | − | + | 20 mg | Local | PH | Old | 20 min. | 10,000 | No |
| | | | | powder | Clinic | | | | | |
| A₂ + A₁ | 19 | − | − | 20 mg powder | Lab | PH | Old | 20 min. | 10,000 | No |
| | | | | | Volunteer | | | | | |
| B | 50 | + | 30 diabetic | 10 mg liquid | Seattle | None | New | 2 min. | 60,000 | Yes |
| | | | 20 normal | | (controlled) | | | | | |
| | | | | | | Yes* | | | | |
| A + B | 78 | | | | | None | | | | |
| Combined | | | | | | | | | | |
| A + B | 78 | | | | | Yes* | | | | |
| Combined | | | | | | | | | | |
| C | 65 | + | 31 diabetes | 20 mg powder | Seattle | None | None | n/a | n/a | n/a |
| | | | 34 normal | 50 minutes | (controlled) | | (whole | | | |
| | | | | prior | | | saliva) | | | |
| C | 65 | + | 31 diabetes | 20 mg powder | Seattle | None | small | 5 | 60,000 | no |
| | | | 34 normal | 2 minutes prior | (controlled) | | | | | |
| C | 65 | + | 31 diabetes | 20 mg powder | Seattle | None | small | 5 | 60,000 | no |
| | | | 34 normal | 7 minutes prior | (controlled) | | | | | |
| C | 65 | + | 31 diabetes | 20 mg powder | Seattle | None | large | 20 | 10,000 | no |
| | | | 34 normal | 12 minutes | (controlled) | | | | | |
| | | | | prior | | | | | | |
| C | 65 | + | 31 diabetes | 20 mg powder | Seattle | Yes* | small | 5 | 60,000 | no |
| | | | 34 normal | 2 minutes prior | (controlled) | | | | | |

| | Pearson Scalar | | | | |
|---|---|---|---|---|---|
| | Correlation | | | Spearman Ordinal | |
| | | | Adj. | Correlation | |
| Study | R | R¹ | R² | R | R² |
| A₁ | .691 | .421 | .376 | .829 | .687 |
| A₂ + A₁ | .868 | .753 | .745 | .882 | .778 |
| B | .542 | .294 | .277 | .610 | .372 |
| | .737 | .543 | .498 | n/a | n/a |
| A + B | .810 | .656 | .652 | .805 | .648 |
| Combined | | | | | |
| A + B | .862 | .743 | .736 | na | n/a |
| Combined | | | | | |
| C | .184 | .034 | .018 | .390 | .152 |
| C | .562 | .316 | .304 | .658 | .433 |
| C | .508 | .258 | .245 | .606 | .367 |
| C | .421 | .177 | .164 | .635 | .403 |
| C | .850 | .722 | .676 | n/a | n/a |

*for known covariates

TABLE 10
Results of ROC analysis using a Blood Glucose threshold value of
126 mg/dl

| | Optimal threshold | | | | |
|---|---|---|---|---|---|
| | for saliva glucose | | | | |
| | based on higher | | | | |
| | sensitivity than | | | | Theta |
| | specificity | | | | (area |
| | Lower | Upper | Sensi- | Specifi- | Under |
| Sample | value | value | tivity | city | curve) |
| All subjects, both | .489 | .531 | .90 | .81 | .82 |
| Studies combined; | | | | | |
| n-78 | | | | | |
| Diabetics, both studies | .434 | .515 | .87 | .63 | .74 |
| combined; n-39 | | | | | |
| Non-diabetics, both | .629 | .629 | 1.00 | .93 | .92 |
| studies combined; n-39 | | | | | |
| All subjects, first | .553 | .676 | 1.00 | .89 | .93 |
| study; n-33 | | | | | |
| Diabetic, first study; | | <1.179 | 1.00 | n/a | n/a |
| n-14 | | | | | |
| Non-diabetic, first | .471 | .676 | 1.00 | .89 | .89 |
| study; n-19 | | | | | |
| All subjects, second | .458 | .524 | .82 | .79 | .78 |
| study; n-45 | | | | | |
| Diabetic, second | .431 | .524 | .81 | .67 | .71 |
| study; n-25 | | | | | |
| Non-diabetic, second | .538 | .616 | 1.00 | .89 | .97 |
| study; n-20 | | | | | |
| Last liquid is none or | .449 | .523 | .70 | .71 | .70 |
| water, second study; | | | | | |
| n-34 | | | | | |
| Last liquid is other | .431 | .550 | 1.00 | 1.00 | 1.00 |
| than water, second | | | | | |
| study; n-11 | | | | | |
| SUMMARY (limits) | .431 | .676 | | | |

### EXAMPLE X

In this Example a subject takes a saliva sampling device of the present invention and places it in her mouth for a period of five minutes. The subject removes the saliva sampling device and measures the amount of glucose in her saliva according to the Examples described above. The amount of glucose in the saliva is used to quantitate the subject's blood glucose level. From this reading the subject then determines if she is at risk for diabetes according to the American Diabetes Association guidelines discussed above.

### EXAMPLE XI

In this Example a subject who is being treated for either Type I or Type II diabetes takes a saliva sampling device of the present invention and places it in her mouth for a period of five minutes. The subject removes the saliva sampling device and measures the amount of glucose in her saliva according to the Examples described above. The amount of glucose in the saliva is used to quantitate the subject's blood glucose level. From this reading the subject then determines if she needs to increase or reduce her blood glucose levels.

### REFERENCES CITED

BAUM, 1993, “Principles of saliva secretion”, Acad Sci., 694:17-23

BORG and BIRK ED, 1988, “Secretion of glucose in human paroitid saliva after carbohydrate intake”, Scand J Dent. Res., 96:551-556.

COHEN, 1988, “Non-enzymatic glycosylation proteins”, Diabet. Ann., 4:469-484

LI, 1994, “Comparing self-monitoring blood glucose devices”, Lab. Med, 25:585-590

REUTERVING et al., 1987, “Salivary flow rate and salivary glucose concentration in patients with diabetes mellitus”, Diabet. Metab., 13:457-462.

What is claimed is:

1. A noninvasive method of diagnosing diabetes comprising:
stimulating salivary gland secretion of saliva into oral fluid;
measuring saliva glucose levels in a subject; and
diagnosing a diabetes disease state in said subject.

2. The method of claim 1, wherein the measuring step further comprises:
collecting a sample of oral fluid;
detecting an amount of glucose in the sample;
detecting an amount of glucose in the sample; and
quantitating a blood glucose level based on the amount of glucose detected.

3. The method of claim 1, wherein said measuring step comprises providing said subject a device for obtaining and measuring glucose levels in a saliva sample.

4. The method of claim 3, wherein said device comprises:
a stimulation means for stimulating salivary gland secretion of saliva into oral fluid;
collection means for collecting a sample of the oral fluid;
detection means operatively connected to said collection means for detecting an amount of glucose in the sample; and
quantitation means operatively connected to said detection means for quantitating blood glucose level based on the amount of glucose detected.

5. The method of claim 4, wherein the device further comprises a housing defining said collection means, said housing containing said stimulation means for release into a buccal cavity.

6. The method of claim 1, wherein said diagnosing step comprises reading a glucose level obtained from said subject and comparing said glucose level to a range of results, whereby said diagnosis of diabetes is made based upon said glucose level.

7. A noninvasive method of monitoring glucose levels comprising:
providing a subject being treated for diabetes;
stimulating salivary gland secretion of saliva into oral fluid;
measuring a saliva glucose level from said patient; and
determining a treatment course of action based on said saliva glucose level.

8. The method of claim 7, wherein said measuring step comprises:
providing said subject a device for collecting a sample of oral fluid;
detecting an amount of glucose in the sample; and
quantitating a blood glucose level based on the amount of glucose detected.

9. The method of claim 8, wherein said device comprises:
a stimulation means for stimulating salivary gland secretion of saliva into oral fluid;
collection means for collecting a sample of the oral fluid;
detection means operatively connected to said collection means for detecting an amount of glucose in the sample; and
quantitation means operatively connected to said detection means for quantitating blood glucose level based on the amount of glucose detected.

10. A method of monitoring blood glucose by:
stimulating salivary gland secretion of saliva into oral fluid;
collecting a sample of the oral fluid;
detecting an amount of glucose in the sample;
quantitating a blood glucose level based on the amount of glucose detected; and
determining a treatment action in view of the blood glucose level quantitated.
